(19) United States
(12) Patent Application Publication
(10) Pub. No.: US 2025/0154498 A1
(43) Pub. Date: May 15, 2025

(54) REAGENTS FOR SUBCELLULAR DELIVERY OF CARGO TO TARGET CELLS

(71) Applicant: ELEVEN THERAPEUTICS LTD, Ramat Gan (IL)

(72) Inventors: Omer ZIV, Cambridge (GB); Andrej ALENDAR, Cambridge (GB); Yaniv ERLICH, London (GB)

(21) Appl. No.: 18/856,820

(22) PCT Filed: Apr. 4, 2023
(86) PCT No.: PCT/EP2023/058794

§ 371 Date: Oct. 14, 2024

(30) Foreign Application Priority Data
Jun. 4, 2022 (EP) 22166923.7
Nov. 7, 2022 (EP) 22205786.1

(51) Int. Cl. C12N 15/10

(52) U.S. Cl. CPC C12N 15/1068; C12N 15/1065

(57) ABSTRACT

The present invention refers to a method for the separation/identification of reagents comprising a compound library, such as DNA-encoded Libraries (DELs), which enter target cells or localize to a desired subcellular compartment of said target cells, by amplifying and modifying the signal of a barcode attached to said reagents thereby significantly increasing the signal to noise ratio and distinguishing the reagents that successfully entered the desired cells or the desired subcellular compartment. Further disclosed herein are reagents, said reagent being able to enter a desired subcellular compartment of a target cell as well as therapeutic applications of said reagents.

### FIELD OF THE INVENTION

The present invention relates to a method for generating and/or identifying reagents able to penetrate a desired subcellular compartment of target cells for delivery of cargo in vitro and in vivo. The invention allows for the identification of successful candidate reagents such as small molecules or aptamers by amplifying and modifying the signal of a barcode attached to said reagents in the desired subcellular compartment thereby significantly increasing the signal to noise ratio and distinguishing the reagents that successfully entered the desired cells or the desired subcellular compartment. Disclosed herein are methods for generating and/or identifying a reagent, said reagent being able to enter a desired subcellular compartment of a target cell as well as therapeutic applications of said reagents.

### BACKGROUND OF THE INVENTION

Most therapeutics require delivery to target tissues or cells. In many cases the amounts of compounds necessary to achieve a therapeutic effect are relatively high since selective delivery is extremely difficult and often relies on targeting the whole body instead, which can result in many side effects. Additionally, biological structures targeted by therapeutics are often within cells. However, cell permeability of many compounds is limited due to biophysical properties. Therefore, there is an unmet demand by the pharmaceutical industry for the identification of reagents and delivery systems that are able to deliver cargo, such as therapeutically active effectors, to specific organs, cells, or subcellular compartments that can be produced efficiently, show high specificity to target cells or subcellular compartments, and low toxicity.

The use of DNA-encoded libraries (DEL) has improved the identification of molecules with certain functions. For this approach, a candidate reagent, typically a small molecule or a peptide, is conjugated with a unique barcode allowing the identification of said reagent through sequencing. This concept has been applied to high-throughput methods by generating candidate libraries comprising millions or billions of different reagents tagged by unique identifying barcode DNA or RNA sequences. Candidate libraries are then incubated with target proteins in vitro. Candidate reagents failing to bind are removed in a subsequent step followed by isolation of nucleic acid barcodes attached to the remaining reagents. Said recovered barcodes are then sequenced in order to identify reagents that successfully bound to the protein

However, while this approach represents a major improvement to conventional small molecule screening approaches, it is inadequate to identify cell-entering molecules due to several limitations: 1) removing unbound reagents is often insufficient to decrease all background signal. This background can be caused by reagents or barcodes that only bind the target cell but fail to internalize or non-specifically attach to the incubation/screening vessel such as plastic or glass dishes 2) Additionally, reagents that internalize through endocytosis but were not able to exit the endosome or reach the desired subcellular compartment of a target cell cannot be distinguished from reagents penetrating the desired compartment since separating different compartments using for instance mechanical approaches is not feasible with this method. Lastly, while DELs have been used extensively in high throughput screens in vitro, in vivo or in cellulo application is not feasible at the moment. Therefore, there is an unmet demand of methods able to identify and/or generate reagents that successfully penetrate a desired subcellular compartment of target cells to, for instance, further deliver cargo molecules attached to said reagents such as therapeutically active effectors.

Reagents particularly suitable to deliver cargo to subcellular compartments of target cells are nucleic acids as their production is cost effective and the technology widely implemented. Additionally, the low immunological properties of nucleic acids limit adverse effects in clinical settings. Chemically modified nucleic acids can be easily attached to most cargo. Therefore, nucleic acids with the ability to bind and enter the cytoplasm or other compartments of a target cell are particularly interesting for therapeutic applications.

Aptamers are short oligonucleotides made of either RNA, DNA, Xeno Nucleic Acids (XNA), RNA/DNA/XNA chimeras, and/or chemically modified nucleic acids. Depending on the nucleic acid base sequence, these short molecules can fold into three dimensional structures and selectively interact with a target molecule such as a protein, lipid, or a small molecule. Certain aptamers bind to a cell membrane receptor and internalize into the cell via receptor mediated endocytosis. While aptamers by themselves can be potent therapeutics, previous studies have further utilized this capability and chemically attached therapeutic oligonucleotides to cell-internalizing aptamers as a means to deliver the payload in a cell-specific manner. However, thus far, the success of this strategy has been limited by the labor-intensive aptamer discovery process in vitro, which requires multiple enrichment steps and has inherent specificity and selectivity limitations.

Historically, aptamer development has utilized Systematic Evolution of Ligands by Exponential Enrichment (SELEX) [Recent reviews: doi: 10.1016/j.talanta.2019.03.015, doi: 10.1016/j.biochi.2018.09.001]. This procedure starts with a candidate library of RNA or DNA oligos bearing a random 20-60 nucleotide long region. Such libraries typically contain about 10 to the power of 15 oligos, which is orders of magnitude smaller than all possible sequence combinations. Consequently, the vast majority of sequences are represented by a single molecule each within the library. The aptamer library is then reacted with a pre-selected receptor such as an immobilized protein or cell type, after which, unbound aptamers are washed away, while bound candidates are eluted and amplified by polymerase chain reaction (PCR). Amplified aptamers are then purified and sequenced to identify the unique sequence of each successful candidate aptamer.

In order to enrich for aptamers with the desired properties, the process above is repeated multiple times by utilizing the successful aptamer candidates from the previous cycle until convergence is achieved.

While this method has been powerful to identify aptamers with high affinity for their selected targets such as proteins, the SELEX platform suffers from sensitivity-selectivity trade-offs similar to those discussed above for DELs: Aptamers that bind non-specifically to the screening vessel or the target cells but fail to internalize, significantly increase the background over the signal of successful candidate aptamers. The sheer number of initial molecules requires multiple selection rounds to eliminate the bulk of non-specific aptamers. Each round involves many PCR amplification cycles, which induce a strong genetic bottleneck and can eliminate highly potent aptamers. As such, the sensitivity of the screen diminishes, resulting in suboptimal selection of aptamers from the initial starting population of aptamers. Moreover, the SELEX approach does not directly select aptamers with the ability to penetrate cells and release their payload into the desired subcellular compartments such as the cytoplasm. However, such functional properties are essential for selective drug delivery.

Further variations of the SELEX basic method were successful to address some of these concerns. For instance, the development of cell-internalization SELEX allows screening for aptamer populations, that are more likely to be internalized by target cells thereby identifying more promising candidates for cargo delivery (Thiel et al., 2012). Only a small subset of candidate aptamers binds target structures and each sequence is present as a single molecule making identification of true positive candidate aptamers highly dependent on contaminating background from non-specifically bound aptamers. Cell-internalization SELEX can reduce some background, which is achieved by stringent washing procedures following aptamer incubation with target cells, which strips bound aptamers from the cell surface while not affecting internalized candidates. Aptamers identified by this method were more likely to internalize into cells and showed some ability to deliver siRNA as cargo to cancer cells. While cell internalization is required for cargo delivery to target cells, the escape of the aptamer or aptamer-cargo chimera to the cytoplasm is essential to fulfill almost all therapeutic functions within the cell. However, cell-internalization SELEX does not screen for aptamers that exit the endosome and suffers similar genetic bottlenecks as described above, which is specifically highlighted by the authors and further indicates a high demand for more suitable aptamers able to not only internalize but also escape the endosome, which has not been successfully addressed within the decade since the publication was released. Lastly, said SELEX method again suffers from sensitivity issues since aptamers that successfully entered the cell are present typically as a single copy and identifying successful candidates is highly challenging, if at all possible, even with the improvement of background reduction. Therefore, amplifying extremely low signal over the background from undesired non-specifically binding aptamers is not feasible with the methods available to this date.

Further reference is made to Alamudi Samira Husen et al., reviewing latest developments of Cell-SELEX as well as US 2016/053265 A1 exploring aptamer internalization mechanisms and their applications in drug delivery.

Therefore, while SELEX is a preferred and well-established method to identify potential aptamer candidates in vitro with binding affinity for selected targets, the current protocols for this method are unable to address either the lengthy and costly process associated with this method, limitation on in vitro screens only, or the failure to identify aptamers that escape from the endosome, essential to unlock the full therapeutic potential of aptamers.

While nucleic acid screening approaches such as SELEX suffer from low sensitivity and inability to identify/generate reagents localizing to desired compartments of target cells, similar constrains apply to small-molecule library screening methods, which encode the chemical identity of the reagent using nucleic acid tags such as DNA Encoded Libraries (DELs).

The use of DELs for the identification of reagents with desired biological functions in recent years has been largely successful. However, the drawbacks of low signal to noise ratios of suitable reagents and un-specifically bound reagents of said libraries as well as the limited ability to screen for reagents which are bioavailable solely in desired (sub) cellular compartments is largely limiting for the industry. The identification or generation of reagents with the ability to deliver cargo such as therapeutics to desired organs, cells, or subcellular compartments, therefore, has the potential to revolutionize medicine and allow more targeted therapies for diseases and conditions previously untreatable due to the lack of reagents targeting desired biological structures.

The underlying objective of the present invention was, therefore, to develop methods allowing to improve the identification and generation of reagents such as aptamers, small molecules, and peptides able to target desired biological structure such as a compartment of a target cell applicable to high-throughput in vitro and in vivo screens, thereby improving the sensitivity of detection methods such as commonly used in DEL- or SELEX-based approaches.

### SUMMARY OF THE INVENTION

The present invention relates to a method for generating and/or identifying a reagent, said reagent being able to enter a desired subcellular compartment of a target cell, comprising the following steps:
a. preparing and/or selecting target cells comprising a polymerase localizing to said desired compartment;
b. preparing a candidate library of reagents comprising or consisting of nucleic acid barcodes recognized by said polymerase;
c. contacting said target cells with said library of reagents, wherein at least a subset of reagents interacts with at least a subset of target cells forming cell-reagent complexes;
d. incubating the cell-reagent complexes thus obtained for a period of time at least sufficient to allow at least a subset of said reagents to enter a desired subcellular compartment of a target cell;
e. amplifying the nucleic acid barcode of said subset of reagents of step d) by said polymerase of step a) within the desired subcellular compartment of the target cells and
f. separating the amplified nucleic acid barcodes of step e) to generate and/or identify said reagent.

In some embodiments, the amplification products of step f) are chemically different from the nucleic acid barcodes of step b), which enables their specific separation. In some embodiments, the amplification products differ from the nucleic acid barcodes by sequence length, sequence orientation, presence of an affinity tag, and/or nucleic acid class.

In some embodiments, the target cells are selected from the group of primary cells, cancer cells, immune cells, organoids, organs, organ-on-a-chip, or combinations thereof.

In certain embodiments, the polymerase is selected from the group of T3, Sp6, T7 RNA polymerase, Phi29 DNA polymerase, Syn5, viral replicases such as alphavirus replicase, or mutants thereof. In a preferred embodiment, said polymerase is T7 RNA polymerase.

In certain embodiments, the candidate library of reagents is selected from the group of DNA-encoded libraries, aptamer libraries, oligonucleotide libraries, polypeptide libraries, peptide libraries, antibody libraries, nanobody libraries, carbohydrate libraries, lipid libraries, or combinations thereof. In some embodiments said candidate library of reagents is a DNA-encoded library. In some embodiments said candidate library of reagents is an aptamer library. In some embodiments said candidate library of reagents is an oligopeptide library.

In some embodiments, the desired compartment according to the invention is any compartment or combinations of compartments of the target cell. In some embodiment, said compartment is selected from the group of cytoplasm, nucleus, Golgi apparatus, endoplasmic reticulum, mitochondria, or combinations thereof.

In a further embodiment, the barcode comprises at least one amplification initialization element, which is useful for in-cell amplification by the polymerase according to the invention. In a preferred embodiment, said amplification initialization element is a T7 promoter. In another embodiment, the barcode is chemically attached to reagents, form electrostatic interactions with the reagent, and/or is encapsulated by the reagent. In another embodiment, the reagent is simultaneously the barcode. In another embodiment, the barcode further comprises a reverse transcription primer site.

In certain embodiments of the method according to the invention it is useful to repeat certain steps of the method to further enrich reagents with desired properties. Therefore, in some embodiments, the method further comprises step g):
i. preparing a new candidate library of reagents from the identified reagents of step f); and
ii. repeating steps a) and c) to f) using said newly prepared candidate library of reagents,
wherein step g) is repeated at least n times,
wherein n is an integer between 0 and at least 1.

In a preferred embodiment, n of step g) denotes an integer of about 0 to about 100, in a more preferred embodiment between about 0 and about 50, in a more preferred embodiment between about 5 and about 50, in a more preferred embodiment between about 5 and about 20, in a more preferred embodiment between about 5 and about 10, in a more preferred embodiment between about 2 and about 5, and in a more preferred embodiment between about 3 and about 10, and in a most preferred embodiment about 1 to about 20.

In certain embodiments, the method further comprising identifying the reagent of step f) thus obtained by sequencing. In another embodiment, the reagent identified in step f) is further modified or optimized using directed evolution, mutagenesis, or chemical modification.

In another embodiment, the incubation of the cell-reagent complexes is carried out at a temperature and for a period of time sufficient to allow the reagents to specifically interact with the desired subcellular compartment of the target cell.

A further aspect of the present invention relates to a delivery reagent comprising a reagent obtainable by the method according to the invention and capable of penetrating a desired subcellular compartment of a target cell. In a preferred embodiment, said delivery reagent is an oligonucleotide, an aptamer, a small molecule, a peptide, a polypeptide, a lipid, a Lipid Nano Particle (LNP), a carbohydrate, or a combination thereof. In another embodiment, the reagent is fused to at least one cargo molecule. In another embodiment, the cargo molecule is a therapeutic agent, diagnostic agent, imaging agent, or toxin.

A further aspect relates to a pharmaceutical composition comprising a therapeutically effective amount of a delivery reagent according to the invention.

Lastly, disclosed herewith is the use of the delivery reagent according to the invention as a medicament or for use in therapy of pulmonary disease.

### Definitions

For convenience, certain terms employed in the specification, examples, and appended claims are collected here.

As used herein, the singular form “a,” “an,” and “the” include plural references unless the context clearly dictates otherwise. For example, the term “target cell” also includes a plurality of target cells and “a compartment” also includes multiple compartments of a target cell.

The terms “polynucleotide” and “nucleic acid” are used herein interchangeably. They refer to a polymeric form of nucleotides of any length: Polynucleotides may have any three-dimensional structure, and may perform any function, known or unknown. The following are non-limiting examples of polynucleotides: coding or non-coding regions of a gene or gene fragment, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

If not stated otherwise, the term “nucleic acid” refers to any nucleic acid such as ribonucleic acid, deoxyribonucleic acid, xeno nucleic acid, single stranded or double stranded.

If not stated otherwise, the term “nucleic acid class” refers to any type of nucleic acid such as ribonucleic acid, deoxyribonucleic acid, xeno nucleic acid.

As used herein, the term “xeno nucleic acids” or “XNAs” are synthetic nucleic acid analogues that have a different phospho-sugar backbone or nucleobases than the natural nucleic acids DNA and RNA.

As used herein, two nucleic acid sequences “complement” one another or are “complementary” to one another if they base pair one another at each position. The term “complement” is defined as a sequence which pairs to a given sequence based upon the canonic base-pairing rules. For example, a sequence A-G-T in one nucleotide strand is “complementary” to T-C-A in the other strand The term “complement” and the phrase “reverse complement” are used interchangeably herein with respect to nucleic acids, and are meant to define the antisense nucleic acid.

As used herein, the term “aptamer” refers to a single stranded and/or double stranded nucleic acid molecule (e.g., ssDNA, ssRNA and/or chimeras of ssRNA and dsDNA) able to specifically bind a structure such as proteins, peptides, nucleic acids, lipids and/or topographic features on a target cell.

The concept of bivalent or multivalent aptamers was proposed and verified in a patent in 1999 by Shi et al., which are defined as modified aptamer structures consisting of two or more identical or different single aptamers, with or without additional structures

As used herein, the term “desired reagent”, “generated reagent”, or “identified reagent” refers to a reagent identified or generated according to the method of the present invention, characterized in that it localizes to a desired subcellular compartment of a target cell.

As used herein, the term “candidate reagent library” or “reagent library” or “candidate library” or “candidate library of reagents” refers to a mixture of reagents or compounds such as, but not limiting, small molecules, proteins, peptides, lipids, polymers or nucleic acids of differing sequence or mixtures thereof, from which to select a desired reagent. A library comprises at least one reagent. A candidate library of reagents is further characterized in that reagents are identifiable by a barcode. In some instances, reagents can be barcodes. For instance, if the reagent library comprises oligonucleotides, said oligonucleotides can serve as both as reagent and barcode, wherein the unique sequence of said reagent/barcode can identify the chemical identity. Non-limiting examples for candidate libraries are aptamer libraries, DNA-encoded libraries (DELs) and oligopeptide library.

As used herein, the term “barcode” refers to a nucleic acid sequence used to identify a reagent. Barcodes can be chemically attached to reagents or form electrostatic interactions with the reagent or can be encapsulated by the reagent. The chemical identity (e.g, structure) of each reagent can be identified by the barcode sequence attached. Reagents can be barcodes by themselves for instance if the reagent comprises a nucleic acid sequence such as aptamers.

As used herein, the term “DNA-encoded library” or “DEL” refers to an embodiment of a candidate reagent library. A DNA-encoded library can comprise a collection of small molecules that are conjugated covalently to DNA tags that serve as identification barcodes.

As used herein, the term “aptamer library” refers to an embodiment of a candidate reagent library. An aptamer library can comprise a collection of nucleic acid molecules, wherein sequences can be different for each molecule. Libraries can have a size as measured in number of molecules from at least one aptamer. The aptamers synthesized in an aptamer library may contain any domain which has a biological function. Non-limiting examples of biological functions of the aptamers described herein include, but are not limited to, localizing to a subcellular compartment, binding to a cell, inducing endocytosis, escaping from the endosome, acting as templates for RNA transcription, binding to, recognizing, and/or modulating the activity of proteins, binding to transcription factors, specialized nucleic acid structure (e.g., Z-DNA, H-DNA, G-quad, etc.), acting as an enzymatic substrate for restriction enzymes, specific exo- and endonucleases, recombination sites, editing sites, or siRNA.

As used herein, the term “contacting” refers to bringing together two or more molecular entities (e.g., reagent library and target cells) such that they can interact with each other. Non-limiting examples for molecular entities are reagents according to the preset invention such as aptamers, proteins, nucleic acids, lipids, and cells.

The term “binding” or “interacting” refers to an association, which may be an association, between two molecules, e.g., between a reagent and target, due to, for example, electrostatic, hydrophobic, ionic and/or hydrogen-bond interactions under physiological conditions.

As used herein, the term “click chemistry” refers to bio-orthogonal reactions known to those of ordinary skill in the art.

As used herein, the term “amplifying” refers to any process or combination of process steps that increases the amount or number of copies of a molecule such as a nucleic acid. Polymerase chain reaction (PCR) is an exemplary method for amplifying of nucleic acids.

As used herein, the term “in-cell amplification” or “in-cell amplifying” refers to the process of making copies of barcodes, wherein the generation of at least one copy of the target barcode is considered as amplified. The copy can be a reverse complement version of the barcode, and can be composed of a different nucleic acid (for example the original barcode is DNA and the copy is made of RNA). Barcodes, which are in-cell amplified according to the invention may be referred to as “amplification products”, “amplification molecules”, or “amplicons”.

As used herein, the term “separating” refers to any process whereby specific subsets of molecules such as nucleic acids, e.g., barcodes attached to reagents localizing to the desired subcellular compartment of a target cell or amplified copies of said barcodes, can be separated from other subsets of molecules. Separating may also refer to the identification of barcode sequences, which have been selectively amplified according to the invention (see also “in-cell amplification”) by means such as, but not limited to, sequencing and analysis of data obtained by methods known in the art.

As used herein, the term “cell reagent complex” refers to an interaction between the reagent and the cell including binding to a structure of the cell surface or transient interactions such as active or passive diffusion through the cell membrane.

As used herein, the term “cargo” refers to any molecule considered for cellular delivery that can be functionally attached to a reagent according to the present invention. Non-limiting examples for possible cargo are nucleic acids, proteins, lipids and small molecules.

As used herein, the term “compartment”, “subcellular compartment” or “cellular compartment” refers to any space enclosed by a cell such as, but not limited by, intracellular space, separated organelles, biological structures such as phase-separated condensates, membranes, or spatially separated structures of a cell.

As used herein, the term “desired (sub) cellular compartment(s)” or “desired compartment(s)” refers to any subcellular compartment or multiple compartments of a cell or multiple cells for which a reagent according to the present invention is sought and for which targeted localization of proteins such as a polymerase is achievable through methods known in the art. A desired subcellular compartment can also refer to multiple compartments. Desired (subcellular) compartment may further refer to a plurality of compartments such as the whole cell or compartments of a selection (sub group) of target cells such as functional structures of multiple target cells (e.g., organs, tissues, or combinations thereof). Non-limiting examples of desired subcellular compartments are cytoplasm, nucleus, Golgi apparatus, endoplasmic reticulum, mitochondria, chloroplast, vacuole, membrane microdomains, nucleolus.

As used herein, a functionally enriched population of reagents refers to a mixture of reagents such as small molecules, aptamers or the barcode attached to the reagents, which is enriched for localizing to a desired subcellular compartment according to the present invention.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient-specific considerations. But a determination of a therapeutically effective amount is within the skill of an ordinarily skilled clinician upon the appreciation of the disclosure set forth herein.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative diseases) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and include the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

The term “amplification initialization element” or “regulatory elements” describes any nucleic acid sequence useful for the recognition of a polymerase according to the present invention in order to initiate amplification.

Unless defined otherwise, the technical and scientific terms used herein have the same meaning as commonly understood by one of ordinary skill in the art to which the invention belongs. Particularly, unless otherwise stated, a term as used herein is given the definition as provided in the Oxford dictionary of biochemistry and molecular biology, Oxford University Press, 1997, revised 2000 and reprinted 2003, ISBN 0 19 850673 2.

### DETAILED DESCRIPTION OF THE INVENTION

A first object of the present invention refers to a method for generating and/or identifying a reagent, said reagent being able to enter a desired subcellular compartment of a target cell, comprising, consisting, or essentially consisting of the following steps:
a) preparing and/or selecting target cells comprising a polymerase localizing to said desired compartment;
b) preparing a candidate library of reagents comprising or consisting of nucleic acid barcodes recognized by said polymerase;
c) contacting said target cells with said library of reagents, wherein at least a subset of reagents interacts with at least a subset of target cells forming cell-reagent complexes;
d) incubating the cell-reagent complexes thus obtained for a period of time at least sufficient to allow at least a subset of said reagents to enter a desired subcellular compartment of a target cell;
e) amplifying the nucleic acid barcode of said subset of reagents of step d) by the said polymerase of step a) within the desired subcellular compartment of the target cells and
f) separating the amplified nucleic acid barcodes of step e) to generate and/or identify said reagent.

The method according to the present invention relies on the specific and autonomous amplification of nucleic acid barcodes attached to the reagents of a candidate library entering the desired (subcellular) compartments within the target cells during the selection screen. Each barcode sequence can be attributed to a specific reagent, thereby making each said reagent identifiable by sequence. Candidates that reach a desired subcellular compartment are selectively amplified in said desired compartment of a target cell by a specific polymerase localizing to said compartment optionally recognizing regulatory elements comprising said barcodes. The in-cell autonomous amplification step highly specifically increases the copy number of barcodes of reagent candidate molecules that entered the desired subcellular compartment, while barcodes of reagents that fail to penetrate said compartments are not amplified. Amplified barcode copies can be separated and detected using sequencing methods known in the art. Amplification of successful candidate barcodes significantly increases the sensitivity of detection over non-specific reagent candidates that were not able to make contact with the polymerase.

Amplification of barcodes that localize to the desired subcellular compartment within a target cell (in-cell amplification) is achieved by providing the barcode with a customizable sequence, which can be recognized by a selected polymerase localizing to said desired compartment, wherein “recognized” refers to the ability of the polymerase to amplify a nucleic acid barcode according to the invention. Thereby, the present invention not only increases sensitivity of the method but also addresses previous limitations present in the art: The specific amplification exclusively in desired compartments reduces false-positive identification of reagents that for instance simply bind a target cell, bind non-specifically to the screening vessel, failed to internalize and/or did not escape the endosome, thereby allowing the identification of reagents able to guide cargo to any accessible cellular compartment.

It has been surprisingly found that the method according to the present invention is able to significantly increase detection sensitivity necessary to generate and/or identify reagents from a starting candidate library of reagents during a screening procedure that are able to enter a desired subcellular compartment of cells (target cells) incubated with said candidate reagents. State-of-the-art methods previously suffered from low sensitivity towards the detection of successful reagent candidates. Therefore, it is especially surprising that the method according to the present invention is able to identify successful candidates in a high throughput-compatible manner by highly specifically increasing the copy number of barcodes corresponding to successful candidate reagents in desired subcellular compartments of target cells.

Significant improvement of identification of reagents localizing to desired compartment of target cells according to the invention is in part a result of the in-cell amplification of barcodes by a specific polymerase. Barcodes colocalizing with said polymerase are selectively amplified. Amplification products increase the signal of barcode sequences present compared to barcode sequences that do not colocalize with the specific polymerase. Accordingly, when barcodes and/or amplification products are separated and identified, barcode sequences that localized to the desired compartment and were amplified are over represented compared to non-localizing barcode sequences. Due to the ability of barcodes to identify the (attached) reagent, the over represented (enriched) barcode sequences allow the separation of said barcode sequences from not overrepresented (depleted) barcodes and identification of encoded reagents that entered the desired compartment.

While the increase in barcode molecules amplified by the polymerase using colocalizing barcodes as a template significantly increases the copy number of amplification products, the amplification products can be also chemically distinguishable from the initial barcode molecule. For instance, the amplification of a DNA barcode by a DNA-dependent RNA polymerase can result in RNA amplification molecules. Said initial DNA barcode molecules can be for instance, selectively digested by Dnase and RNA recovered. Amplification products can be, for instance distinguishable from their barcode template by size, chemical modifications, sequence orientation, and/or incorporation of affinity tags. This further allows the selective removal or separation of template barcodes together with barcodes that did not colocalize with the polymerase from amplification products. Accordingly, following the removal/separation of the initial library barcodes, amplification products remain that can be analyzed further reducing background noise.

Some nucleic acids such as aptamers have intrinsic properties allowing delivery of cargo due to their three-dimensional shape. However, identifying sequences able to penetrate specific cellular compartments of target cells requires screening a massive number of different sequences and high sensitivity for the identification of the few expected candidates providing such properties.

It has been surprisingly found that the method according to the present invention is able to generate and identify reagents such as aptamers and small molecule populations from a starting candidate library according to the invention that show a high degree of endosomal escape to the cytoplasm or other compartments of cells incubated with said candidates (target cells). This is especially surprising since state-of-the-art approaches such as SELEX, DEL screens, or other methods known in the art were previously unable to screen reagent libraries for the ability to escape from the endosome due to sensitivity restrictions as well as failure to resolve spatial resolutions such as individual cellular compartments.

In some embodiments, the method according to the present invention enables the specific amplification of cell-penetrating and endosome-escaping reagent barcodes by the target cells themselves during the reagent selection screen. The in-cell amplification step e) according to the present invention highly specifically increases the copy number of reagent candidate molecules that escaped from the endosome and localized to desired compartments expressing the polymerase, while aptamers that cannot bind target cells, fail to internalize, cannot escape the endosome and/or localize to undesired compartments are not amplified. In addition, the polymerase may generate a reverse complement template (amplicon, amplification product) that contains unique and known sequences that are not in the original reagent library, thereby allowing for their selective amplification. This allows increasing the signal of reagents with desired properties over undesired reagent sequences present in the library (see also FIG. 1). In some embodiments, the amplification products of the nucleic acid barcodes by said polymerase of step (a) are chemically different from the parental template barcodes, which enables their specific identification.

It has been surprisingly found that the combination of (i) in-cell amplification and (ii) selective readout of the in-cell amplification products (amplicons) promises a superior sensitivity-selectivity balance than traditional SELEX or methods to screen DEL for cellular delivery. The present invention is designed to select only reagents such as aptamers or DEL capable of both cell entry and endosomal escape (to enable in-cell amplification during the screen), two crucial properties of delivery reagents. Furthermore, due to the strong in-cell amplification, endosomal escape barcodes can be detected for weeks after their introduction into cells. Such prolonged duration can facilitate the selection of delivery reagents with high stability suitable for slow drug release.

State-of-the-art SELEX or DEL screening approaches require multiple cycles of purification of successful candidate reagents and re-screening said candidates again to enrich for reagent binding a desired target partially due to a high number of false-positive candidates associated with the high background and low sensitivity of the method. These cycles make such methods a lengthy, cost-intensive, and complicated approach for the identification of reagents. Therefore, it is especially surprising that the method according to the present invention significantly reduces the number of cycles necessary to identify highly specific cellular delivery candidates, thereby decreasing cost, labor and increasing specificity of the functionally enriched barcode population for endosomal escape and localization to desired compartments in target cells.

The method according to the present invention comprises several steps. In a first step a), a target cell comprising a polymerase localizing to a desired compartment for which a reagent that penetrates said desired subcellular compartment is sought, is selected and/or prepared.

In some embodiments, a “target cell,” as used herein, denotes an in vivo or in vitro eukaryotic cell, a prokaryotic cell (e.g., bacterial or archaeal cell), cells that comprise an organ, an organ on a chip, an organism, or a cell from a multicellular organism cultured as a unicellular entity (e.g., a cell line), which eukaryotic or prokaryotic cells can be, or have been used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation.

In some embodiments, the target cell is a prokaryotic cell. In some embodiments, the cell is a bacterial cell. Non-limiting examples of bacteria include Aspergillus, Brugia, Candida, Chlamydia, Coccidia, Cryptococcus, Dirofilaria, Gonococcus, Histoplasma, Klebsiella, Legionella, Leishmania, Meningococci, Mycobacterium, Mycoplasma, Paramecium, Pertussis, Plasmodium, Pneumococcus, Pneumocystis, Pseudomonas, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, and Clostridium botulinum. In some embodiments, the cell is a eukaryotic cell. In some embodiments, the cell is an animal cell (e.g., a mammalian cell). In some embodiments, the cell is a human cell. In some embodiments, the cell is from a non-human animal, such as a mouse, rat, rabbit, pig, bovine (e.g., cow, bull, buffalo), deer, sheep, goat, llama, chicken, cat, dog, ferret, or primate (e.g., marmoset, rhesus monkey). In some embodiments, the cell is a parasite cell (e.g., a malaria cell, a leishmanias cell, a Cryptosporidium cell or an amoeba cell). In some embodiments, the cell is a fungal cell, such as, e.g., Paracoccidioides brasiliensis.

In some embodiments, the target cell is a cancer cell (e.g., a human cancer cell or a patient-derived cancer cell). In some embodiments, the cell is from any cancerous or pre-cancerous tumor. Non-limiting examples of cancer cells include cancer cells from the bladder, blood, bone, bone marrow, brain, breast, colon, esophagus, gastrointestine, gum, head, kidney, liver, lymph nodes, lung, nasopharynx, neck, ovary, pancreas, prostate, skin, stomach, testis, tongue, or uterus. In addition, the cancer may specifically be of the following histological type, though it is not limited to these: neoplasm, malignant, carcinoma, carcinoma, undifferentiated, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, malignant, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, malignant, branchiolo-alveolar papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, pagets disease, mammary, acinar cell carcinoma, adenosquamous carcinoma, adenocarcinoma w/squamous metaplasia, thymoma, malignant, ovarian stromal tumor, malignant, thecoma, malignant, granulosa cell tumor, malignant, and roblastoma, malignant, sertoli cell carcinoma, leydig cell tumor, malignant, lipid cell tumor, malignant, paraganglioma, malignant, extra-mammary paraganglioma, malignant, pheochromocytoma, glomangiosarcoma, malignant melanoma, amelanotic melanoma, superficial spreading melanoma, malig melanoma in giant pigmented nevus, epithelioid cell melanoma, blue nevus, malignant, sarcoma, fibrosarcoma, fibrous histiocytoma, malignant, myxosarcoma, liposarcoma, leiomyosarcoma, rhabdomyosarcoma, embryonal rhabdomyosarcoma, alveolar rhabdomyosarcoma, stromal sarcoma, mixed tumor, malignant, mullerian mixed tumor, nephroblastoma, hepatoblastoma, carcinosarcoma, mesenchymoma, malignant, brenner tumor, malignant, phyllodes tumor, malignant, synovial sarcoma, mesothelioma, malignant, dysgerminoma, embryonal carcinoma, teratoma, malignant, struma ovarii, malignant, choriocarcinoma, mesonephroma, malignant, hemangiosarcoma, hemangioendothelioma, malignant, kaposi's sarcoma, hemangiopericytoma, malignant, lymphangiosarcoma, osteosarcoma, juxtacortical osteosarcoma, chondrosarcoma, chondroblastoma, malignant, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, soft tissue sarcoma, ganglioneuroblastoma, neuroblastoma, retinoblastoma, olfactory neurogenic tumor, meningioma, malignant, neurofibrosarcoma, neurilemmoma, malignant, granular cell tumor, malignant, malignant lymphoma, Hodgkin's disease, Hodgkin's lymphoma, paragranuloma, malignant lymphoma, small lymphocytic, malignant lymphoma, large cell, diffuse, malignant lymphoma, follicular, mycosis fungoides, other specified non-Hodgkin's lymphomas, malignant histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia.

In some embodiments, the target cell is an immune cell (e.g., a human immune cell or a patient-derived immune cell). As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

In some embodiments, the target cell is susceptible for viral infection or is infected by a virus. For example, in some embodiments, the virus is SARS-COV-2, SARS-COV-1, HIV, hepatitis A, hepatitis B, hepatitis C, herpes virus (e.g., HSV-1, HSV-2, CMV, HAV-6, VZV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV, dengue virus, papillomavirus, molluscum virus, poliovirus, rabies virus, JC virus, or Ebola virus.

In certain embodiments, target cells according to the invention are multiple cells and/or tissues of an organism or all cells of an organism such as an animal or human. In some embodiments, the target cells denote the entirety of an organism such as a (genetically engineered) mouse. In some embodiments, the target cells are a tissue comprising multiple cell types such as an organ, or an artificial tissue such as an organoid. In some embodiments, the target cells are an organ-on-a-chip. In some embodiments, the target cells are cultured in a screening vessel such as a plastic or glass dish. In some embodiments target cells are grown in culture media suitable for growth. In some embodiments, the target cells are growing within the natural or artificially altered environment of the tissue and/or organism.

In a preferred embodiment the target cell is a eukaryotic cell, preferably a cell of mammalian origin.

In a preferred embodiment, target cells are selected from the group of primary cells, cancer cells, immune cells, organoids, organs, organ-on-a-chip, or combinations thereof.

In some embodiments, target cells are selected from a combination of different cell types as listed above.

In some embodiments, the target cells used is selected or prepared to express a polymerase able to in-cell amplify a barcode according to the invention.

In some embodiments, said polymerase is of cellular or foreign origin.

In some embodiments, genetic sequences encoding said polymerase can be permanently introduced into the genome of said target cell using genome engineering approaches such as TALEN, Zinc finger nucleases or CRISPR/Cas-related methods or by retroviral infection, such as lentivirus.

In a further embodiment polymerase can be delivered to the target cell as a protein or nucleic acids, such as DNA or RNA, coding for the respective polymerase.

In some embodiments, the polymerase is a nucleic acid dependent nucleic acid polymerase. In some embodiments, the polymerase is selected from the list of DNA dependent DNA polymerase, DNA dependent RNA polymerase, RNA dependent DNA polymerase or RNA dependent RNA polymerase. In some embodiments the polymerase is a replication complex made of more than one subunit (e.g., viral replication machinery).

In preferred embodiments the polymerase is selected from the list of T7 RNA polymerase, T3 RNA polymerase, SP6 RNA polymerase, Phi29 DNA polymerase, Syn5, viral replicases such as alphavirus replicase, mutants thereof.

It has been surprisingly found that certain polymerases, in particular T7 RNAP, with DNA-dependent RNA activity can be utilized for in-cell amplification in order to generate reverse complement copies of barcodes that contain an accessible priming sequence for reverse transcription, which is inaccessible (the priming sequence) in the original barcode sequence (in sense direction), thereby allowing the exclusive separation of barcodes amplified by said polymerase by reverse transcription and subsequent identification of said barcode sequences. Therefore, in an even more preferred embodiment the polymerase according to the present invention is a T7 RNA polymerase (T7 RNAP). In some embodiments, a DNA-dependent RNA polymerase such as T7 polymerase transcribes the DNA barcodes into RNA. In some embodiments, the parental DNA barcodes can be specifically eliminated by methods known in the art, which allows selective identification of the in-cell amplification RNA progenies (amplification products). In some embodiments, said T7 polymerase can be mutated to modulate its activity, such as, but not limited to, the Ser43Tyr mutation in T7 RNAP.

Expression of the polymerase according to the invention in a desired subcellular compartment (such as the nucleus, the cytosol, etc.) of the target cell can facilitate the selection of reagents capable of entering said subcellular compartment. A desired subcellular compartment (or desired compartment) is any biological structure of the target cells or combinations thereof for which a penetrating reagent is sought.

Reagents with the ability to distribute not within the whole target cell or random compartments of the target cell but rather distribute/localize to separate and selected (=desired) (sub) cellular compartment(s) are in particularly high demand by the industry. The ability to select the target/desired compartment of a cell enables precision cargo delivery to biological structures such as whole organelles, receptors, membranes, or even phase-separated condensates by utilizing reagents obtainable by the method according to the invention.

Surprisingly, it has been discovered that tethering the polymerase according to the invention (e.g., trapping the polymerase enzyme in a compartment using protein tags or tethers such as nuclear localization sequences or other methods known in the art) to the selected compartment(s) according to the invention or preventing localization of said polymerase to undesired compartments, allows selective in-cell amplification of reagents colocalizing with said polymerase, thereby amplifying barcode sequences of reagents with the ability to enter said desired compartment(s). The identification of such reagents with the ability to enter desired compartments has been laborious and economically unfeasible with state-of-the-art methods. In particular, while several reagents able to enter or associate with target cells, using known methods to the skilled person, were identified, the majority of such reagents entering a cell are trapped in endosomes thereby preventing delivery of cargo molecules to biological structures in need thereof and reducing bioavailability. Accordingly, reagents able to enter a desired compartment are indistinguishable from such reagents associated with the target cell in other ways (e.g., localizing to an undesired compartment or bind to said cells un-specifically) with approaches utilizing known methods. The physical isolation of the desired compartment in order to remove such unwanted reagents is laborious, economically unfeasible for larger screens, not possible for many compartments, and generally results in high contamination of unwanted sequences due to the low copy number of barcodes. The method according to the invention, therefore, represents a significant improvement over known methods and allows even the identification or generation of reagents localizing to compartments previously unable to isolate with common methods known to the skilled person.

The desired subcellular compartment according to the invention may refer to any cellular compartment or a group of compartments that comprise a target cell. In some embodiments, the desired subcellular compartment is the whole target cell. In some embodiments, the desired subcellular compartment according to the invention refers to at least one subcellular compartment. In a preferred embodiment the desired compartment is selected from the list of cytoplasm, nucleus, Golgi apparatus, endoplasmic reticulum, mitochondria, or combinations thereof. In certain embodiments, a desired subcellular compartment may refer to the outer and/or inner membrane of the target cell or its organelles.

While the method according to the invention enables the identification or generation of reagents according to the invention for homogenous target cell populations such as cell lines known in the art, the method is additionally particularly suitable for the identification of reagents that localize to structural formations of organisms such as organs. For instance, in some embodiments target cells may refer to an organisms such as a (genetically modified) mouse, wherein the desired compartment may refer to selected subcellular compartments of a plurality of cells forming biological structures such as organs of said organism (e.g., lung, heart or combinations thereof). In some embodiments, the desired subcellular compartment is selected by expressing the polymerase according to the invention in a selected set of cells such as an organ by methods known in the art such as tissue specific expression using cre/loxP or cooption of tissue specific regulatory elements (e.g., promoters). In some embodiments the desired compartment is the subcellular compartment of a selection of cells (sub group) comprising the target cells forming functional structures such as separate organs of live animals or other structurally organized cell and tissue formations formed by target cells such as for instance, but not limited, to lungs, kidneys, hearts, or gonads.

In-cell amplification of reagent barcodes is dependent on contact between the barcode and said polymerase as described above. Changes in the localization of said polymerase, therefore, can be used to selectively amplify reagent barcode subsets localizing to desired subcellular compartments. Accordingly, the desired subcellular compartment can be selected by targeted localization of the polymerase according to the invention to desired subcellular compartments. Localization of said polymerase can be directed to individual compartments by common methods known in the art such as localization tags (e.g., Nuclear Localization Sequence (NLS), Nuclear Export Sequence (NES)).

In a preferred embodiment of the present invention said polymerase is ubiquitously abundant within all compartments of the target cell, in a more preferred embodiment said polymerase proteins are present in at least one cellular compartment or combinations thereof.

In a more preferred embodiment said polymerase proteins are present in at least one of the compartments selected from the list of cytoplasm, nucleus, Golgi apparatus, endoplasmic reticulum, mitochondria, or combinations thereof.

In a further step b) a suitable reagent library is generated. Said library comprising at least one reagent attached to at least one nucleic acid barcode able to identify said reagents by the barcode sequence(s) or its amplification products according to the invention. In some embodiments library size as measured in number of reagent molecules comprising unique barcodes is at least 10 to the power of 1 reagents, more preferred at least 10 to the power 5 reagents, even more preferred at least 10 to the power 10 reagents and most preferred at least 10 to the power 15 reagents. In some embodiments library size is between about 10 to the power of 1 and 10 to the power of 15 reagents, more preferred between 10 to the power of 2 and 10 to the power of 10, more preferred between 10 to the power of 3 and 10 to the power of 8, more preferred between 10 to the power of 3 and 10 to the power of 7, more preferred between 10 to the power of 3 and 10 to the power of 6 and more preferred between 10 to the power of 4 and 10 to the power of 6.

The barcode according to the present invention comprises a unique nucleic acid sequence able to identify attached reagents and optionally at least one amplification initialization element recognized by said polymerase. In a preferred embodiment said element is an origin of replication and/or a promoter transcription initiation site such as a T7 promoter sequence.

In some embodiments, the barcode consists of a payload that can identify the reagent and an error detecting code to validate the correctness of the payload sequence, such as a CRC32 or a Reed-Solomon code.

In some embodiments, the reagent according to the present invention is a therapeutic agent selected from the group consisting of tyrosine kinase inhibitors, kinase inhibitors, biologically active agents, biological molecules, radionuclides, adriamycin, ansamycin antibiotics, asparaginase, bleomycin, busulphan, cisplatin, carboplatin, carmustine, capecotabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, melphalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), 5-fluorouracil and derivatives, radionuclides, polypeptide toxins, apoptosis inducers, therapy sensitizers, enzyme or active fragment thereof, and combinations thereof.

In some embodiments the reagent according to the present invention is selected from the list of small molecules, peptides, proteins, lipids, polymers, lipid nanoparticles (LNPs), polymers and nucleic acids such as aptamers or combinations thereof.

In some embodiments the candidate library is a DNA-encoded library (DEL).

In some embodiments the candidate library is an aptamers library. Aptamer libraries comprise unique nucleic acid sequences that can serve as barcodes and/or amplification initiation elements analogous as the barcodes described according to the present invention. In some embodiments the reagent is an aptamer and the aptamer simultaneously comprises a barcode and/or amplification initiation element according to the present invention (see also FIG. 2).

In order to stabilize RNA aptamers and improve delivery vehicle in vivo, the ribose 2′ hydroxyl on the RNA can be replaced with a fluor group, making the RNA unrecognizable by cellular and serum nucleases. In some embodiments RNA aptamers according to the present invention comprises modified nucleotides. In a preferred embodiment, RNA aptamers comprise modifications of the ribose 2′ hydroxyl on the RNA backbone selected from the list of 2′Ome nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In a more preferred embodiment, RNA aptamers comprise 2′Ome nucleotides, 2′-deoxy-2′-fluoro (2′F) nucleotides modifications, phosphonothioate, and mixtures thereof.

In a further next step c) target cells from step a) are contacted with the prepared reagent library from step b). Said reagent library is contacted with the target cell in a way that allows interaction of said reagents with the target cells wherein at least a subset of the reagents interacts with at least a subset of the target cells forming cell-reagent complexes. Cell-reagent complexes in this context can be any interaction between reagent and cell such as binding of the reagent to the cell membrane, binding to a cell membrane receptor, or active or passive diffusion through the cell membrane.

Reagent libraries may be contacted with the target cells according to the invention using approaches known by the skilled person in the arts such as but not limited to applying the library of reagents to the culture medium of target cells. In some embodiments target cells are contacted with the reagent library by dissolving said library in the culture medium of the target cells and applying said culture medium to said target cells.

In certain embodiments of the invention the target cells are an organism such as a mouse, wherein said target cells are contacted with the reagent library by injection, transfusion (=parenteral administration) and/or feeding of said library of reagents. In some embodiments the reagent library is contacted with the target cells by preparing a composition obtained or obtainable by dissolving the library of reagents in a suitable pharmaceutically acceptable carrier, diluent, or excipient thereof, wherein the composition thus obtained is applied by parental administration.

Compositions adapted for parenteral administration may include aqueous and non-aqueous sterile injection solutions comprising antioxidants, buffers, bacteriostatics and solutes, by means of which the formulation is rendered isotonic with the blood of the organism to be contacted; and aqueous and non-aqueous sterile suspensions, which may comprise suspension media and thickeners. The formulations can be administered in single-dose or multidose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules, and tablets.

In some embodiments the ratio of reagents comprising the candidate library according to the invention to target cells (reagents:cells) is between about 1:10 to the power of 15 and 10 to the power of 15:1. In a preferred embodiment, said ratio is between about 10 to the power of 13:1 and 10 to the power of 3:1. In a preferred embodiment, said ratio is between 10 to the power of 5:1 and 10 to the power of 2:1.

The reagent library can be contacted with target cells under any condition conductive to form cell-reagent complexes. The condition includes, but is not limited to, for examples, a controlled period of time, an optimal temperature (e.g., 37° C.), and/or an incubating medium (e.g., target cell culture medium), etc. In some embodiments the reagent library is contacted with the target cell in vitro or in vivo. In some embodiments the reagent library is contacted by injection into an organism. In a preferred embodiment the reagent library is contacted with the target cell under physiological conditions to allow binding of aptamers or small molecules to the target cells.

In a subsequent step d) the reagent library is incubated with the target cells to give the reagents the opportunity to interact with the cell surface and to allow entering of the desired subcellular component.

In certain embodiments the candidate reagent library is incubated with the target cells for extended periods of time allowing a subset of reagents to reach desired subcellular compartments in the target cell, thus allowing the degradation of said reagents that show low long-term stability and thereby further selecting and screening for reagents that show high chemical stability within the desired compartment. In some embodiments, the period of time at least sufficient to allow at least a subset of said reagents to enter a desired compartment of a target cell are between about 1 second and about 5 days, between about 30 minutes and about 4 days, between about 1 hour and about 3 days, between about 1.5 hours and about 24 hours, or between about 1.5 hours and about 2 hours. In some embodiments, the period of time of incubation may be, for example, 10 min, 15 min, 30 min, 45 min, 1 hour, 2 hours, 4 hours, 6 hours, 8 hours, 12 hours, 16 hours, 24 hours, 2 days, 3 days, 4 days, 5 days, 6 days, 1 week, 1 month, 1 year or 10 years. In a preferred embodiment, the period of time at least sufficient to allow at least a subset of said reagents to enter a desired compartment of a target cell is between about 1 h and 72 h, between 5 h and 48 h, between 24 h and 40 h, and more preferred between 24 h and 30 h.

In a subsequent step e) the barcodes of reagents that successfully reached the desired cellular compartment are autonomously amplified within said compartment of the host cell by said polymerase expressed in the host cell (in-cell amplification). Said barcodes are amplified only in the cellular compartment to which said polymerase localizes. Consequentially, copy number of barcodes attached to reagents entering the desired compartment are increased with high spatial control. Amplification of barcodes is initiated by the polymerase for instance by recognition of an amplification recognition element sequence comprising the barcode or reagent.

In some embodiments, the amplification according to step e) of the method according to the present invention can lead to either transcription, replication, or rolling circle amplification of barcode sequences generating barcode amplicons with a reverse complement sequence to the original barcodes (see also FIG. 2, 5).

In step f) of the method according to the invention, following the intracellular amplification of the barcode according to step e), said amplified barcodes are separated from other barcode subsets, thereby identifying and generating a reagent being able to enter a desired subcellular compartment of a target cell. In some embodiments, said reagent is a functionally enriched population of reagents.

Following the amplification of barcode sequences of reagents that were in-cell amplified by the polymerase according to the invention, said in-cell amplification products are separated from barcode sequences. Separation may refer to the identification of the chemical identity of reagents corresponding to the amplified barcode sequences by methods known in the art. For instance, separation may refer to the sequencing of the amplification products alone or in mixture with further nucleic acids, such as unamplified barcode molecules, and analyzing enriched sequences followed by identifying reagents based on the sequence of the enriched barcode sequences.

In certain aspects, provided herein is a functionally enriched population of reagents generated by a method provided herein. In certain embodiments, the reagent population is characterized by a more than 1.1-fold functional enrichment (e.g., a more than 1.5-fold functional enrichment) compared to the reagents in the library of candidate reagents before enrichment. In some embodiments, the functional enrichment is between about 1.1 and about 1,000,000, between about 5 and about 1,000, between about 10 and about 1,000, between about 100 and about 1000-fold, and between about 1,000 and about 50,000-fold. In some embodiment the function is localization of the reagent to a desired subcellular compartment.

In some embodiments, separation may further comprise physically separating amplification product molecules form other nucleic acids such as the initial barcode molecules comprising the reagent library and/or barcode molecules that did not amplify. Separating for example can comprise selectively immobilization of the nucleic acid subset on magnetic beads or gel electrophoresis of the desired nucleic acid size or specifically degrading the parental library or by specifically amplifying the in-cell amplification progenies using specific primers that do not amplify the parental library.

In some embodiments, separation comprises purifying amplification products using methods known in the art such as total RNA or DNA extraction from target cells. In some embodiments of the method according to the present invention, a subset of reagents comprising the reagent library that were unable to internalize are separated from the other reagent subsets. In certain embodiments separation comprises washing off barcodes that bind to the target cell surface using washing procedures known in the art. In certain embodiments separation is achieved by washing off barcodes that bound target cells but did not internalize using stringent washing procedures known in the art, thereby stripping the cell surface off bound barcodes. In a further embodiment the separation is achieved by digesting the barcode of the reagents that did not bind to or interact with the target cell surface using nucleases such as endo- or exonucleases such as DNAses or RNAses. In some embodiments, separation of amplified barcodes comprises digesting the initial barcodes comprising the barcode library followed by isolation of the amplified barcode molecules. Isolation of barcodes may refer to the simultaneous isolation of amplification products according to the invention together with other nucleic acids present in the target cells. In some embodiments, amplification products are selectively separated from unamplified barcodes and/or the in-cell amplified nucleic acid barcodes of step e) further in-vitro amplified to generate and/or identify said reagents according to the invention.

In some embodiments, the DNA barcodes of reagents such as aptamers or DEL that failed to be amplified inside the target cells are degraded using DNA specific nuclease, while the RNA products of in-cell amplification are insensitive to this degradation. In some embodiments, the DNA barcodes contain specific labile bases that can be selectively degraded post in-cell amplification. In some embodiments, the DNA barcodes contain an affinity tag, such as biotin, which facilitates its depletion post in-cell amplification via affinity selection (for example by using Streptavidin coated beads).

In some embodiments the in-cell amplification generates a reverse complement copy of the original barcode. In an embodiment of the method, said barcode further comprises a unique priming site, which is a sequence corresponding to a priming sequence such as a reverse transcription primer (RT primer) site. In some embodiments of the invention, the amplification product is an RNA. This amplification product can serve as the basis for an in vitro strand-specific Reverse Transcription PCR (RT-PCR) amplification. In this process a reverse transcription primer, which sequence corresponds to the unique priming site of the barcode amplicon, is provided and enables the priming of the RT reaction. This approach exclusively targets the products of the in-cell amplification (barcode amplicon), and further enhances the copy number of the barcode reverse complement sequence, thereby increasing the signal over the original reagent barcode (see also FIGS. 2 to 6).

In the case of rolling circle amplification, the in-cell amplification products differ also in size from the original reagent barcode, which can further be used to separate the in-cell amplification progenies of barcodes, via size exclusion chromatography (see also FIGS. 5 and 6).

In some embodiments, separated barcodes of reagents that successfully penetrated a desired compartment according to step f) can be further amplified and purified prior to identification to further increase sensitivity and exclude undesired barcodes or other nucleic acids contributing to background signal.

In some embodiments, separated barcodes according to step f) are analyzed to identify and/or generate reagents according to the present invention by methods known in the art such as sequencing.

Reagents identified with the method according to the present invention are characterized by the properties of interacting with a target cell and penetrating a desired subcellular compartment. In some cases, it might be useful to further optimize the candidate reagents thus obtained to further identify especially suitable candidate and remove false-positive candidates in an additional screening. In this context, reagents identified by the method according to the present invention can serve as the foundation for a new reagent library. In a further embodiment of the method, said new library can be screened again. In this process the same target cell or any newly selected or generated target cell according to step a) is selected and the process described in steps c) to f) is repeated. In some embodiment, said additional screening is repeated at least once, in a preferred embodiment said additional screening is repeated at least once to 20 times, in a more preferred embodiment said additional screening is repeated at least once to 10 times and in a most preferred embodiment said additional screening is repeated at least once to 5 times.

In some embodiments it is useful to identify candidate reagents that not only localize to a desired compartment but additionally do not localize to one or more different compartments. In an alternative embodiment said reagents are obtainable or identifiable by screening the said newly generated candidate library again in target cells specifically amplifying reagent barcodes of candidates in compartments that are undesirable. Thereby a subset of reagents for said newly generated reagent library can be identified that not only penetrates the desired compartment but also undesired compartments. Said new subset can be subtracted from the initial successful candidate library to further enrich reagents penetrating only the desired compartment.

### Libraries

Libraries according to the present invention refer to a mixture of structurally diverse reagents or compounds such as, but not limiting, small molecules, proteins, peptides, lipids, polymers or nucleic acids of differing sequence or mixtures thereof, from which to select a desired reagent. A library comprises at least one reagent. Non-limiting examples for candidate libraries are aptamer libraries or DNA-encoded libraries (DELs).

Candidate libraries of reagents according to the invention are obtainable according to methods known in the art. For instance, reagents can be fused to barcodes by chemical, enzymatic or electrostatic means. DNA encoded libraries can be produced via a split-pool strategy, and DNA-templated macrocycle libraries are obtainable by allowing multiple enrichment rounds per screen as further described in Usanov et al., 2019 (doi.org/10.1038/s41557-018-0033-8). Barcodes may serve as barcode and reagent at the same time. Said barcode/reagent libraries are obtainable by methods known in the art such as split-pool strategies, wherein a plurality of different barcode/reagent sequences is generated. BARCODES

Appropriate design of nucleic acid barcodes according to the present invention is critical for the success of in-cell amplification according to the invention. Recognition of a barcode by a polymerase localizing to the desired sub-cellular compartment according to the invention, may require a polymerase-identification sequence such as a promoter. For instance, in-cell amplification by T7 polymerase may require a T7 promoter sequence. The nucleic acid barcode can comprise single stranded nucleic acids, double stranded nucleic acids, or can be a hybrid with stretches of single and double stranded sequences. Nucleic acids can be any natural or chemically modified nucleic acids known in the art such as but not limited to DNA, RNA, XNA or combinations thereof.

Depending on the selected barcode design, identification of in-cell amplified copies may require optimized separation strategies known in the art by the skilled person. For instance, transcripts generated from single stranded RNA or DNA barcodes may be identifiable by strand specific sequencing and/or PCR since the transcript sequence is distinguishable from the template sequence of the original barcode. This is advantageous since the barcode introduced into the target cell itself is not identified and does not contribute to background signal. However, the use of double stranded nucleic acid barcodes can result in increased background signal since the amplified transcripts of the barcode may be indistinguishable from the originally introduced barcodes. Therefore, in the case of contaminants such as barcodes sticking to the cell surface, said barcodes can introduce unwanted background noise. However, strategies can be applied to reduce said background noise. For instance, when DNA barcodes are used according to the invention and barcode sequences are in-cell amplified by an RNA polymerase such as T7 polymerase, transcripts can be separated from the barcode molecules by DNA digestion of the original barcode molecules, pull down of original barcode molecules and/or other known methods in the art.

Candidate library of reagents according to the invention comprise or consist of nucleic acid barcodes, allowing identification of the chemical identity of the members of said library. Accordingly, in some embodiments, said barcode can identify the chemical identity of the attached reagent.

### INDUSTRIAL APPLICATION

The identified reagents localizing to a desired compartment of a target cell according to the present invention can be used in various applications and methods. Reagents can have intrinsic therapeutical activity and can be used to target subcellular compartments to exert a biological function such as binding target structures e.g., proteins. Reagents identified according to the present invention can be further chemically fused to cargo in order to deliver said cargo to desired compartments of target cells. In this function, the reagent can be designed to target specific cells and/or cellular compartments for cargo delivery by customizing the method in accordance with the present invention. These properties allow the targeted delivery of cargo to many structures previously thought to be undruggable.

Another aspect of the present invention, therefore, refers to a delivery reagent comprising a reagent obtainable by the method according to the present invention and capable of penetrating a desired subcellular compartment of a target cell. In a more preferred embodiment, said reagent is selected from the list of small molecules, peptides, proteins, lipids, polymers, lipid nanoparticles (LNPs), polymers and nucleic acids such as aptamers or combinations thereof. In a preferred embodiment, the present invention relates to a delivery reagent wherein said delivery reagent is an aptamer or a small molecule. In an even more preferred embodiment said reagent is a small molecule.

In a preferred embodiment said delivery reagent is chemically fused to at least one cargo molecule. In a preferred embodiment of the invention cargo is considered any molecule considered for cellular delivery, preferably selected from the list consisting of nucleic acids, proteins, lipids, small molecules, more preferably RNA or DNA, and most preferably siRNAs, gRNAs, lncRNAs, tRNAs, mRNAs, piRNAs and shRNAs.

Delivery reagents can be fused to cargo in many ways known in the art. For example, nucleic acid cargo can be covalently attached to the reagent by click chemistry. Additionally, multiple identical or different molecules of cargo can be attached to the delivery reagent. For instance, but not limiting, two molecules of heterologous cargo can be attached to a delivery reagent such as an aptamer obtainable by the method according to the present invention (see also FIG. 8).

In some embodiments, multiple reagents obtainable by the method of the present invention can be chemically attached. For instance, multiple aptamers can be bivalently or multivalently conjugated (see also FIG. 8) or multiple small molecules can be attached to different parts of the same cargo.

Another object of the present invention refers to a method to deliver cargo to target cells comprising the following steps:
a) providing a target cell in need of cargo delivery;
b) providing the delivery reagent according to the invention;
c) chemically attach said cargo to said reagent and
d) contacting said target cells with the reagent thus obtained.

The delivery reagent according to the present invention can be used as transfection reagent able to deliver cargo such as nucleic acids to selected cells.

The delivery reagent according to the present invention can be further used as a medicament or therapeutical. For instance, the reagent can be fused or conjugated to therapeutic drugs or other effector molecules.

The concept of cargo delivery by biological reagents such as antibodies specifically targeting structures on target cells is well known. In this process, antibodies are conjugated to effector molecules forming so called immunoconjugates, which have been intensively used as therapeutics to treat medical conditions such as cancer.

In some embodiments the present invention relates to a delivery reagent comprising a reagent obtainable by the method according to the invention and capable of penetrating a desired subcellular compartment of a target cell. In a further embodiment the present invention relates to a delivery reagent comprising a nucleic acid capable of binding a target cell, inducing endocytosis and internalization into said target cell and subsequent endosomal escape, wherein the nucleic acid is an aptamer obtainable according to the method of the present invention.

It has been surprisingly found that reagents according to the present invention are able to deliver cargo to target cells and, in contrast to immunoconjugates, show high release rates to cellular compartments such as the cytoplasm allowing the possibility to target structures previously out of reach.

The present disclosure provides a pharmaceutical composition comprising a therapeutically effective amount of a delivery reagent provided by the invention, optionally attached to at least one cargo molecule, or a salt thereof, and a pharmaceutically acceptable carrier or diluent. Relatedly, the present disclosure provides a method of treating or ameliorating a disease or disorder, comprising administering the pharmaceutical composition to a subject in need thereof.

Administering a therapeutically effective amount of the composition to the subject may result in: (a) an enhancement of the delivery of cargo to a disease site or a subcellular compartment of a target cell relative to delivery of the cargo alone; or (b) an enhancement of target clearance resulting in a decrease of at least 10%, 20%, 30%, 40%, 50%, 60%, 70%, 80%, or 90% in a blood level of the target of the reagent, e.g., a protein; or (c) an decrease in biological activity of the target of the reagent, e.g., a protein, of at least 10%, 20%, 30%, 40%, 50%, 60%, 70%, 80%, or 90%.

Administering a therapeutically effective amount of the pharmaceutical composition according to the invention can be achieved by any means known such as intravenously, subcutaneously, intraperitoneally, orally, or through inhalation.

The disease or disorder can include without limitation those disclosed herein. The disease or disorder may include without limitation COVID19, Influenza, Breast Cancer, Alzheimer's disease, bronchial asthma, Transitional cell carcinoma of the bladder, Giant cellular osteoblastoclastoma, Brain Tumor, Colorectal adenocarcinoma, Chronic obstructive pulmonary disease (COPD), Squamous cell carcinoma of the cervix, acute myocardial infarction (AMI)/acute heart failure, Chron's Disease, diabetes mellitus type II, Esophageal carcinoma, Squamous cell carcinoma of the larynx, Acute and chronic leukemia of the bone marrow, Lung carcinoma, Malignant lymphoma, Multiple Sclerosis, Ovarian carcinoma, Parkinson disease, Prostate adenocarcinoma, psoriasis, Rheumatoid Arthritis, Renal cell carcinoma, Squamous cell carcinoma of skin, Adenocarcinoma of the stomach, carcinoma of the thyroid gland, Testicular cancer, ulcerative colitis, or Uterine adenocarcinoma.

Delivery reagents such as aptamers according to the present invention are particularly useful for treatment of pulmonary diseases caused by infection. Aptamers can be modified to increase chemical stability and functionally conjugated to cargo such as siRNAs.

A further embodiment, therefore, refers to a delivery reagent obtainable according to the present invention as a medicament or for use in therapy of pulmonary disease. In particular, aptamers obtainable according to the present invention can be selected for selective delivery of therapeutics to for instance the lung epithelia, which is commonly targeted by many pathogens such as viruses e.g., SARS-COV-2. The present invention therefore discloses pharmaceutical compositions useful to treat pulmonary diseases. In a preferred embodiment, the pharmaceutical embodiment comprises an aptamer fused to therapeutical RNA such as a siRNA useful to treat COVID19.

The invention will be explained in more detail by virtue of the examples set forth herein below. While at least one exemplary embodiment is presented, it should be appreciated that a vast number of variations exist. It should also be appreciated that the exemplary embodiments are only examples, and are not intended to limit the scope, applicability, or configuration in any way. Rather, the forgoing description will provide those with ordinary skill in the art with the essential characteristics of this invention for implementing at least one exemplary embodiment, it being understood that various changes may be made without departing from the scope as set forth in the appended claims.

### BRIEF DESCRIPTION OF THE FIGURES

FIG. 1 shows a schematic representation of a possible embodiment of the method. Target cells expressing the polymerase T7 transgene are challenged with a reagent library of 10 to the power of 15 aptamers. Aptamers that enter the cell and escape to the cytoplasm are amplified by the T7 polymerase, improving the signal to noise ratio. The cells are then thoroughly washed to remove the excess of non-specific aptamers. Finally, the extracted RNA undergoes a selective RT-PCR that targets only the negative strand T7 amplification products.

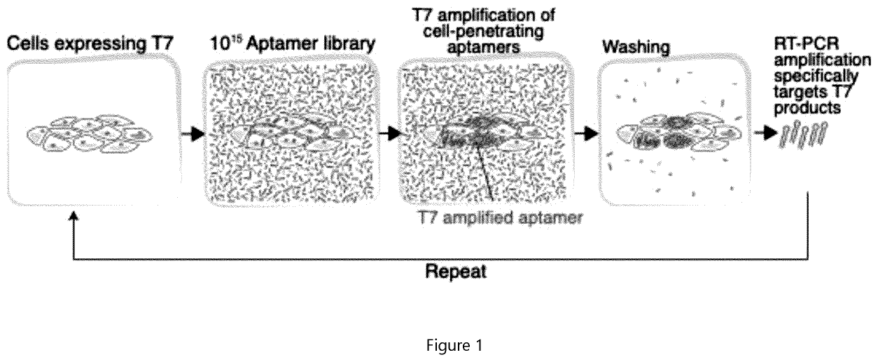

FIG. 2 shows a further aspect of an embodiment of the method, an example for the design of a possible reagent, said reagent being an aptamer. The aptamer serves as reagent as well as barcode in this example. The aptamer has a T7 promoter [left] and an RT primer binding site [right]. Cell-penetrating aptamers are amplified by the T7 RNA polymerase expressed by the cells, creating a reverse complement copy [middle]. The RT-PCR targets the RT primer binding site on the reverse complement strand of the aptamer for strand-specific amplification. Amplified sequences can be further separated and analyzed by for instance sequencing.

FIG. 3 illustrates an embodiment of the present invention and exemplarily describes a possible separation strategy of amplified barcodes according to step f). In this example, the barcode consists of a dshoDNA (double stranded homoduplex DNA)/ssRNA chimera aptamer, wherein the DNA part comprises a T7 promoter and the RNA part further comprises a RT primer binding site. The barcode sequence is represented by the overall sequence of the aptamer, which in this context represents the reagent. Following in-cell amplification according to step e) using a T7 polymerase expressed ubiquitously and localizing to the cytoplasm, the amplicons are RNA molecules with a reverse complement sequence in regards of the original barcode. Said amplicons can be selectively reverse transcribed yielding a single strand DNA molecule, which can be optionally further purified before analyzing the sequence of the aptamer in order to identify said reagent according to the present invention, wherein said reagents represents an aptamer able to bind, internalize and escape from the endosome of a target cell.

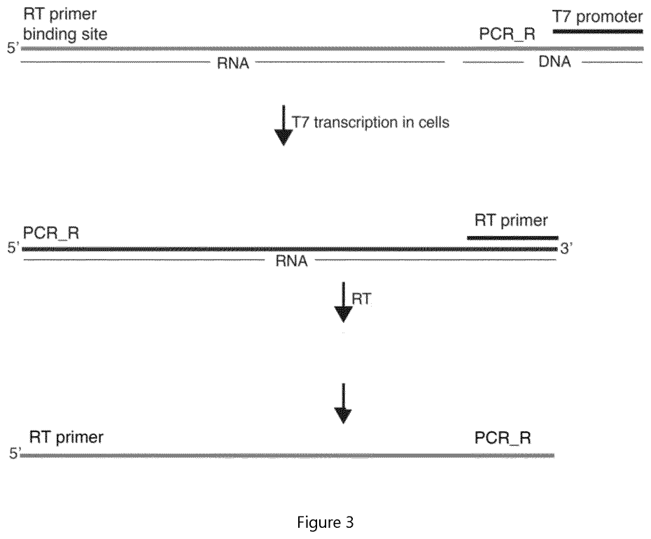

FIG. 4 illustrates a possible embodiment of the present invention in which the reagent according to the present invention relates to an aptamer analogous to FIG. 3. Said aptamer being in-cell amplified by T7 RNA polymerase (T7 RNAP). The amplicon is further reverse transcribed using a RT primer binding the RT binding site contained within the aptamer sequence. Reverse transcribed DNA molecules can be further purified using methods known in the art in order to purify said barcodes and increase signal to noise ratio in subsequent analysis methods such as sequencing, by removing remnants of non in-cell amplified aptamers.

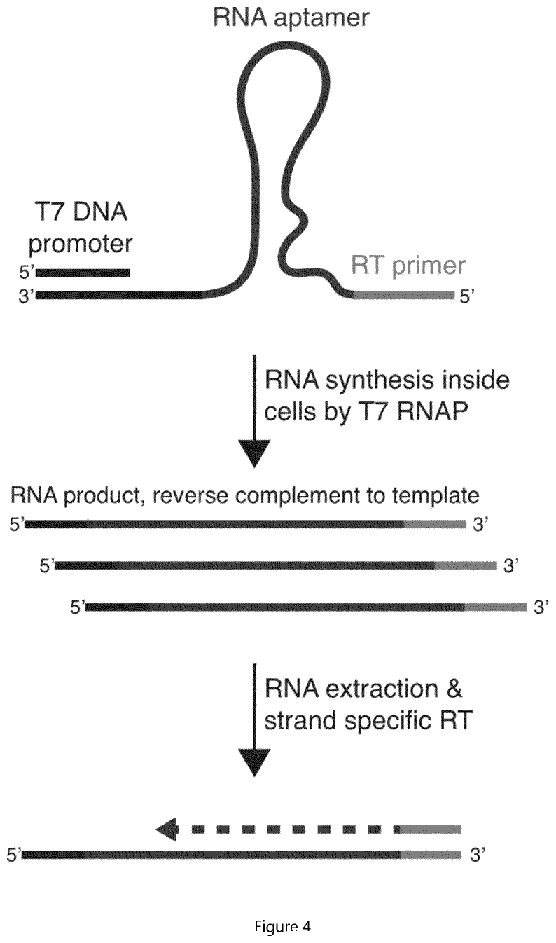

FIG. 5 shows a further embodiment of the present invention. The reagent according to the invention can be a circular aptamer, comprising an amplification initiation site such as a T7 promoter. In-cell amplification according to step e) can be rolling-circle amplification resulting in long stretches of repetitive sequences in reverse complement orientation to the aptamer sequence. Said repetitive sequences can be RNA molecules. DNA aptamers that failed to be amplified can be digested using Dnase. Large fragments resulting from the rolling circle T7 amplification can be separated from low molecular weight aptamers that failed to amplify and further selectively reverse transcribed. Reverse transcribed DNA fragments can be analyzed according to methods known in the art such as sequencing.

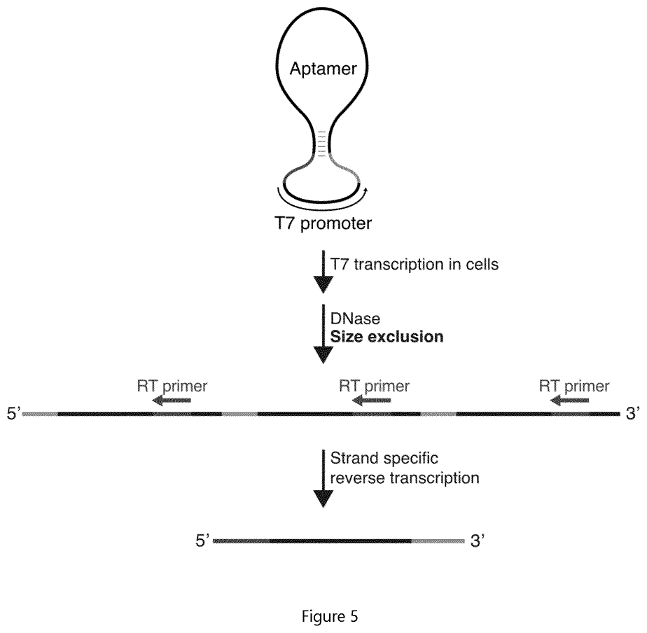

FIG. 6 shows an example for a circular DNA aptamer according to the present invention comprising a barcode sequence, an amplification initiation element, a T7 promoter, which is recognized by a T7 RNAP according to the invention. In-cell amplification results in the generation of repetitive RNA stretches in reverse complement orientation in regards to the original aptamer sequence. The reverse complement amplicons reveal an accessible RT primer site, which is bound by RT primers in order to initiate reverse transcription. Following reverse transcription, generated DNA fragments can be isolated and analyzed according to methods known in the art such as sequencing.

FIG. 7 shows a further embodiment of the present invention. Shown is an example for a delivery reagent obtainable according to the method of the present invention further conjugated to cargo. The reagent can be comprised of an aptamer identified from a reagent library according to the method. Said reagent can be conjugated to cargo such as oligonucleotides (e.g., siRNAs). The delivery reagent fused to cargo can be useful as a therapeutic reagent delivering cargo to a desired subcellular compartment of a target cell by means of the reagent identified according to the method of the present invention.

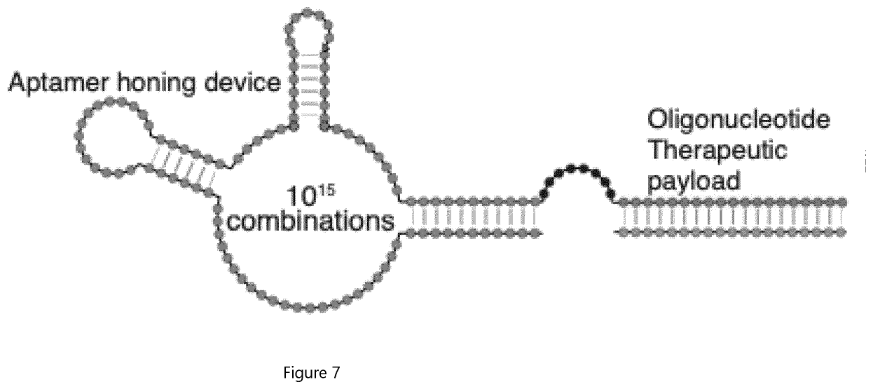

FIG. 8 illustrates further embodiments of the present invention. Delivery reagents identified according to the present invention such as aptamers can be fused to cargo such as nucleic acids in many ways known in the art. For example, nucleic acid cargo can be covalently attached to the aptamer, bind through sticky ends, conjugated by click chemistry. Furthermore, multiple aptamers can be bivalently or multivalently conjugated. Additionally, multiple identical or different molecules of cargo can be attached to the delivery reagent. For instance, but not limiting, two molecules of heterologous cargo can be attached to a delivery reagent such as an aptamer obtainable by the method according to the present invention.

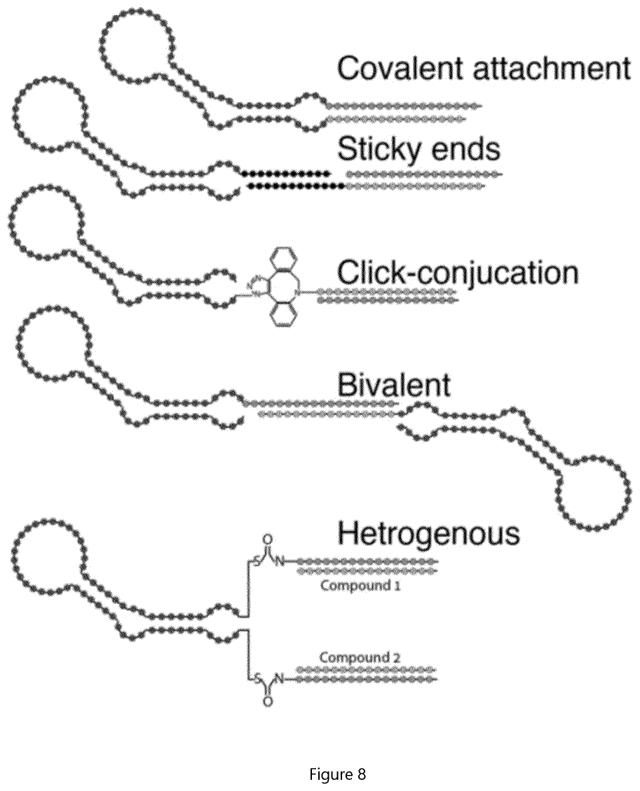

FIG. 9 illustrates the result obtained for testing the ability of T7 RNA polymerase (RNAP) to utilize a single stranded RNA aptamer covalently linked to a double stranded DNA T7 promoter as a template for in vitro transcription. Shown is a TBE-Urea gel with sizes of nucleic acid fragments shown for in vitro transcription at 30° C. and 37° C. respectively.

FIG. 10 shows cell cytometry results of cells ectopically expressing T7 polymerase and a GFP-reporter gene under the control of the T7 amplification sequence (T7 promoter). T7 RNA polymerase transcription of the GFP DNA plasmid in the cytoplasm of the cells results in GFP fluorescent signal. The GFP plasmid contains an Internal Ribosomal Entry Site (IRES) to circumvent the lack of CAP on the resulting GFP mRNA. Additionally, a negative control of cells, not expressing T7 polymerase was included (lower panel). GFP positive cell populations were gated and shown is the percentage of cells expressing GFP.

FIG. 11 shows fluorescence microscopy images of two cell lines expressing T7 RNA polymerase (T7 RNAP) and a GFP-reporter gene under the control of the T7 amplification sequence (T7 promoter) and IRES. Signal is shown in white.

FIG. 12 shows the results obtained for testing the ability of T7 RNA polymerase (RNAP) to utilize a single stranded 2′fluoro modified RNA aptamer as a template for highly effective transcription in vitro. Shown is a TBE-Urea gel, one star denotes the expected size of the 2′fluoro RNA-DNA template. Two-stars denotes the expected size of the resulting RNA product (of note, the T7 amplification product doesn't include the promoter and is therefore shorter).

FIG. 13 shows results obtained for testing the ability of T7 RNA polymerase (RNAP) to utilize a circular single stranded RNA aptamer as a template for in vitro transcription. Shown is a TBE-Urea gel with sizes of nucleic acid fragments shown for in vitro transcription. Star denotes high molecular weight concatemeric RNA products resulting from rolling circle amplification by T7.

FIG. 14 illustrates the results of a qPCR experiment indicating the differences in delta Ct values of in-cell amplification by cells either expressing a T7-controlled circular barcode sequence (in this case aptamer) and T7 polymerase or a negative control expressing the T7-controlled circular barcode only. Shown is the quantification for three biological replicates.

FIG. 15 shows an embodiment of a design of a nucleic acid barcode attached to a reagent forming a DNA-encoded library (DEL) member according to the invention. The barcode features a polymerase promoter (T7) attached to the barcode of a DNA encoded library (DEL) to enable signal amplification inside cells that express the cognate polymerase (e.g., cells expressing T7 polymerase). The DEL library can carry an affinity tag (e.g., biotin) to enable its removal following extraction from cells, and before reverse-transcription and sequencing of the T7 products. Dnase treatment can be used to further specifically degrade the parental DEL, resulting in specific measurement of the in-cell RNA progenies in subsequent sequencing steps.

FIG. 16 illustrates a further embodiment of a barcode according to the present invention. Depicted is a double stranded barcode design useful for the identification of reagents localizing to desired compartments (top). Further shown are results of a qPCR experiment, in which said barcode is amplified within the cell by T7 polymerase (bottom). X-axis depicts presence (+) or absence (−) of T7 polymerase (T7 RNAP). Y-axis shows Ct value of detected nucleic acid for three biological replicates.

FIG. 17 illustrates a further embodiment of a barcode according to the present invention. Depicted is a single stranded barcode design with a partial double stranded T7 promoter (T7) useful for the identification of reagents localizing to desired compartments further comprising a sequencing adapter (top). Further shown are results of an experiment, in which said barcode is amplified within the cell by T7 polymerase. X-axis depicts presence (+) or absence (−) of T7 polymerase (T7 RNAP). Y-axis shows Ct value for qPCR of detected nucleic acid for three biological replicates.

FIG. 18 illustrates a further embodiment of the invention. Depicted is a barcode design and identification strategy according to the method of the invention. A partial double and partial single stranded barcode design is shown, which allows in-cell amplification according to the invention. Amplification products of barcodes able to enter the desired subcellular compartment (compartment expressing the polymerase) are then separated from the original barcode molecules and sequenced.

FIG. 19 illustrates the results of an experiment exploring the ability of distinguishing cell-internalizing barcodes from barcodes without this ability using the method of the present invention. Top of the figures shows experiment utilizing a single stranded barcode, while bottom shows experiment for double stranded barcode. X-axis depicts cell entry optimization of barcode (+) or absence of such optimization (−). Y-axis shows Ct value for qPCR of detected nucleic acid for three biological replicates.

FIG. 20 illustrates the results of an experiment exploring the ability of distinguishing barcodes able to localize to a desired subcellular compartment from barcodes without this ability using the method of the present invention. X-axis depicts cell entry optimization of barcode (cell entry: +) or absence of such optimization (cell entry:−) as well as bioavailability (bioavailability: +) or no availability (bioavailability:−). Y-axis shows Ct value for qPCR of detected nucleic acid for three biological replicates. Upper panel shows the detection of in-cell amplification products. Lower panel shows the detection of the original DNA library in each sample.

FIG. 21 illustrate the results of an experiment testing the ability of the method of the present invention to identify barcodes from a library according to the invention able to localize to a subcellular compartment. Plots depicted show spiked-in positive controls (black) enrichment and negative controls (gray) barcodes without a cell-internalizing reagent. Only barcodes that showed an enrichment of more than 1 are shown. Left shows sequencing of in-cell amplification products, while right shows sequencing of original barcode molecules following extraction from cells.

FIG. 22 illustrate the results of an experiment testing the ability of the method of the present invention to identify barcodes from a library according to the invention able to localize to a subcellular compartment. Plots depicted show spiked-in Cholesterol-TEG conjugated positive controls (chol), spiked-in cy3 conjugated negative controls (cy3), and non-conjugated negative controls (naked) as well a library of non-conjugated barcodes (bulk). T7 on: shows sequencing of in-cell amplification products, while T7-off shows sequencing of original barcode molecules following extraction from cells.

### EXAMPLES

### Example 1

### T7 In Vitro Transcription of RNA Aptamers

The improvement of sensitivity for the identification of reagents that are able to localize to a desired compartment within a target cell according to the present invention relies in part on the efficient amplification of barcodes attached to said reagents within the cell. Barcodes such as aptamers are necessary to identify the reagents according to the present invention. In order to test the efficiency of amplification of said barcodes, the ability of T7 RNA polymerase (RNAP) to utilize a single stranded RNA aptamer as a template for in vitro transcription was assessed.

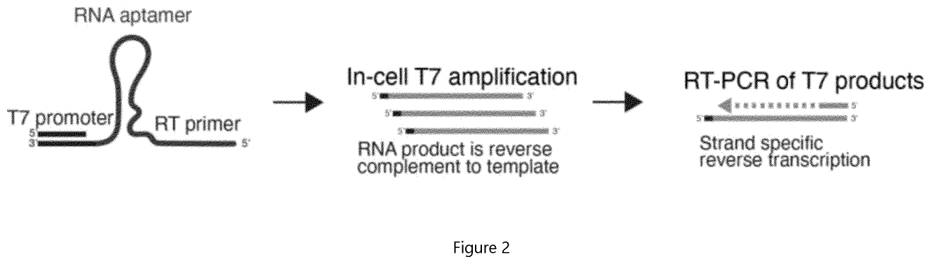

To do so a 42 nucleotide long single stranded RNA fused on its 3′ to a 36 nt long DNA was used as a template for in vitro T7 transcription. The 5′ end of the DNA was single stranded while the 3′ end of the DNA was a double stranded T7 promoter (see also FIG. 2). In vitro transcription was carried out by HiScribe™ T7 High Yield RNA Synthesis Kit (NEB) according to the manufacturer recommendations, in either 30 or 37 Celsius degrees, after which the resulting RNA was assessed for quantity by Qubit™ RNA BR Assay Kit (Invitrogen) and visualized on a Novex™ TBE-Urea Gels, 15% (Invitrogen).

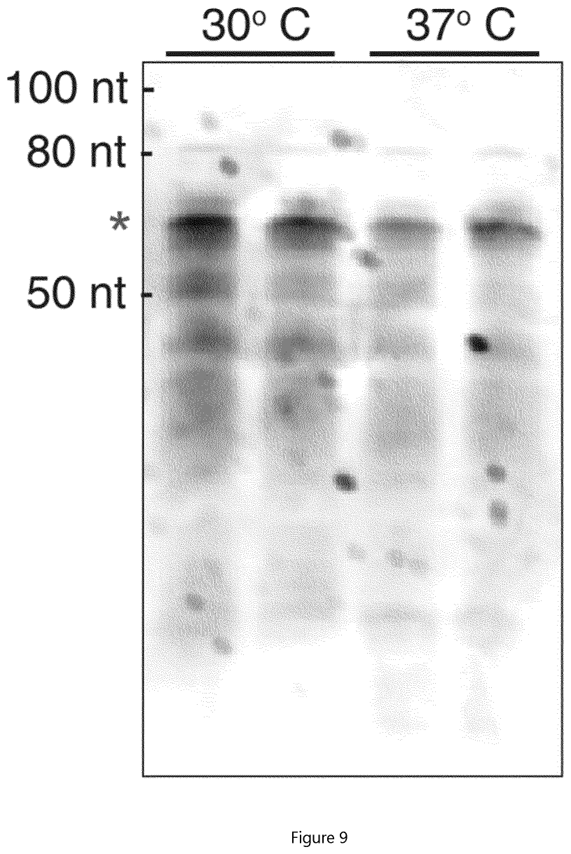

The data presented in FIG. 9 show that T7 RNAP is capable of utilizing a single stranded RNA aptamer as a template for highly effective transcription in vitro. Star denotes the expected size of the resulting RNA product.

### Example 2

### In-Cell Amplification of RNA Aptamer

While aptamers according to the present invention were able to be transcribed in vitro, the method according to the present invention requires amplification within the target cell and optionally in a desired subcellular compartment. Therefore, the ability of in-cell amplification of barcodes such as aptamers was assessed.

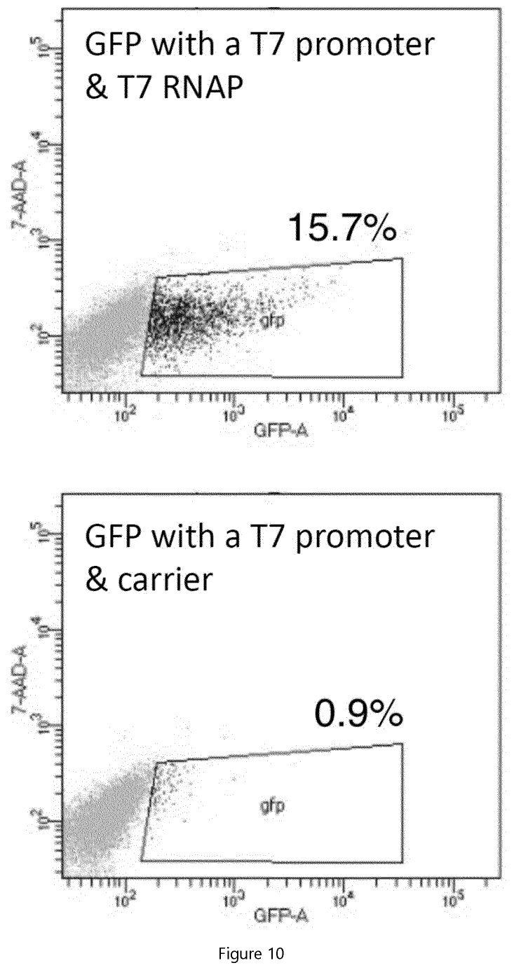

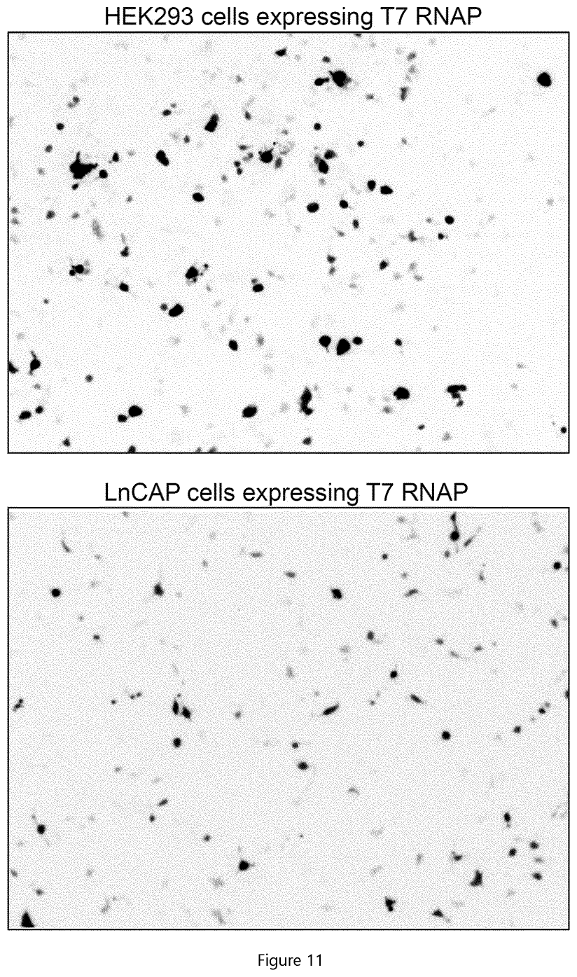

HEK293 cells were co-transfected with (i) a plasmid encoding a GFP reporter gene under the control of a T7 promoter and an Internal Ribosomal Entry Site (IRES), and (ii) a T7 RNAP mammalian expression plasmid or a carrier plasmid that do not encode for T7 RNAP as a control. All transfections were performed using Lipofectamine™ 3000 Transfection Reagent (Invitrogen) according to the manufacturer recommendations. GFP expression was assessed 30 hours following transfection by Cell Cytometry (FIG. 10) or fluorescence microscopy (FIG. 11). Fluorescence microscopy was additionally performed for a second cell line (LnCAP) expressing a T7 RNAP expression plasmid, along with a plasmid encoding a GFP reporter gene under the control of a T7 promoter.

The data presented in FIGS. 10 and 11 show that T7 RNAP ectopically expressed in human cells is catalytically active and can transcribe RNA from a T7 promoter inside cells. These results illustrate that in-cell amplification (e.g., of a barcode such as aptamer according to the present invention) is highly efficient and suitable to increase copy numbers of barcodes thereby increasing signal to noise ratio over background.

### Example 3

### T7 In Vitro Transcription of Circular DNA Aptamer

In some embodiments of the present invention, the barcode or aptamer can be a circular nucleic acid such as a single stranded circular DNA or RNA sequence. The objective of this example was to test the ability of T7 RNAP to utilize a single strand DNA circle as a template for rolling circle amplification in vitro according to the present invention.

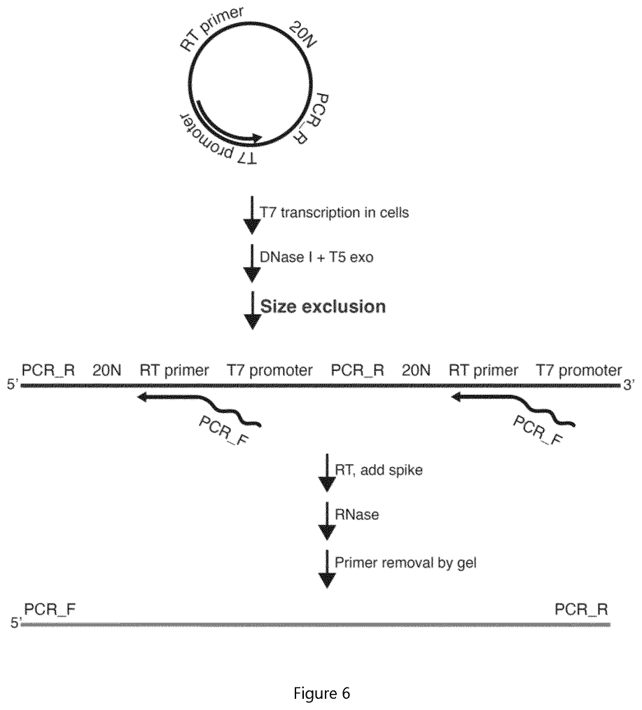

A 70 nucleotide long single stranded DNA circle annealed to aT7 promoter (as shown in FIG. 6) was used as a template for in vitro T7 transcription by HiScribe™ T7 High Yield RNA Synthesis Kit (NEB) according to the manufacturer recommendations. Following removal of the DNA template by TURBO™ Dnase (Invitrogen), the resulting RNA was visualized on a Novex™ TBE-Urea Gels, 15% (Invitrogen).

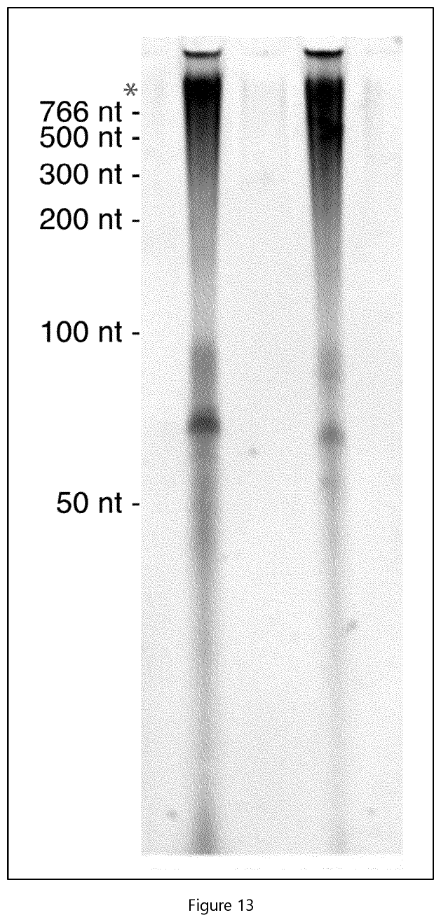

The results are presented in FIG. 13. The data show that T7 RNAP is capable of utilizing a single strand DNA circle as a template for rolling circle amplification in vitro. Star denotes high molecular weight concatemeric RNA products resulting from rolling circle in vitro amplification by T7.

### Example 4

### T7 In Vitro Transcription of 2′Fluoro Modified RNA Aptamer

In order to stabilize RNA aptamers and make them a better delivery vehicle in vivo, the ribose 2′ hydroxyl on the RNA can be replaced with a fluor group (2′F), making the RNA unrecognizable by cellular and serum nucleases. In order to test the ability of amplification of said barcodes made of 2′fluoro modified RNA, the ability of T7 RNA polymerase (RNAP) to utilize a single stranded 2′fluoro modified RNA aptamer as a template for in vitro transcription was assessed.

To do so, a 42 nucleotide long single stranded RNA fully modified with 2′fluoro in all positions was fused on its 3′ to a 36 nt long DNA and was used as a template for in vitro T7 transcription. The 5′ end of the DNA was single stranded while the 3′ end of the DNA was a double stranded T7 promoter (see also FIG. 2). In vitro transcription was carried out by HiScribe™ T7 High Yield RNA Synthesis Kit (NEB) according to the manufacturer recommendations in 37 Celsius degrees, after which the resulting RNA was assessed for quantity by Qubit™ RNA BR Assay Kit (Invitrogen) and visualized on a Novex™ TBE-Urea Gels, 15% (Invitrogen).

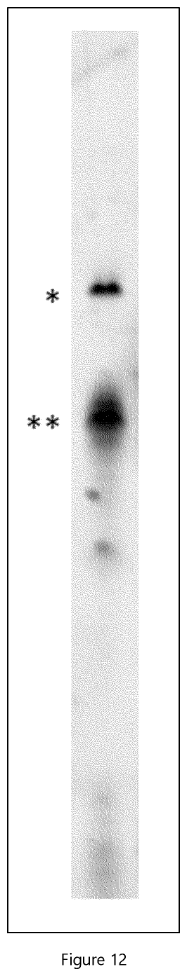

The data presented in FIG. 12 show that T7 RNAP is capable of utilizing a single stranded 2′fluoro modified RNA aptamer as a template for highly effective transcription in vitro. Star denotes the expected size of the 2′fluoro RNA-DNA template. Two-stars denotes the expected size of the resulting RNA product (of note, the T7 amplification product doesn't include the promoter and is therefore shorter).

### Example 5

### In-Cell Amplification of Circular DNA

Following the demonstration that T7 polymerase is able to amplify circular barcodes resulting in concatemeric RNA products, in this example the ability of in-cell amplification using said circular barcodes was evaluated. To do so, in cell T7 rolling circle amplification of circular single stranded DNA aptamers, and strand-selective in vitro amplification of the T7 products was performed. Transcription of circular DNA can result in repetitive transcripts of the target nucleic acid forming long stretches. Said stretches can be size selected by gel chromatography thereby purifying amplification products from their parental circular template for further analysis.

The single stranded DNA circular aptamer shown in FIG. 6 was co-transfected into HEK293 cells along with a plasmid encoding for T7 RNAP, or with a control plasmid, using Lipofectamine™ 3000 Transfection Reagent (Invitrogen) according to the manufacturer recommendations. Following 30 hours incubation, cellular RNA was extracted using a Quick-RNA Miniprep Kit (Zymo Research). DNA remnants were digested using TURBO™ Dnase (Invitrogen). In order to further eliminate remnants of the original circular aptamer library, the extracted RNA was loaded on a Novex™ TBE-Urea Gels, 10% PAGE (Invitrogen), and RNA larger than 350 nt long was excised for further analysis. In cell T7 products were reverse transcribed using a strand specific primer and SuperScript™ III Reverse Transcriptase (Invitrogen). The resulting cDNA was used as a template for quantitative PCR reactions targeting the in cell T7 amplification products of the aptamers.

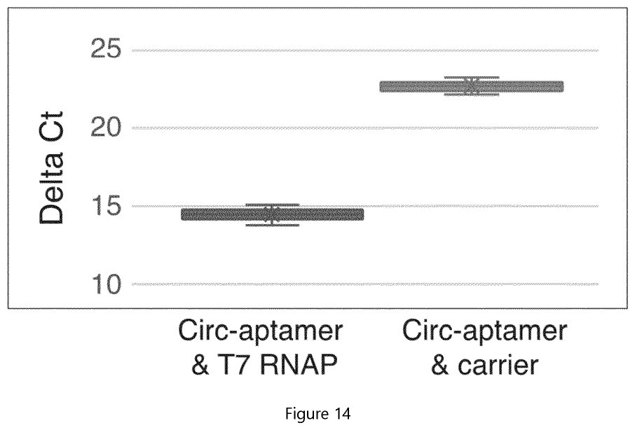

The results of the qPCR are shown in FIG. 14. Average qPCR cycle thresholds of three biological replicates are shown. The data demonstrate effective T7 rolling circle amplification of a single stranded circular aptamer inside human cells, and selective in vitro amplification of the in-cell T7 products, with 8 PCR cycles difference from control cells without T7 RNAP. This shows the ability to select and further amplify aptamers that reached the cell cytoplasm and become available to interact with cytoplasmic proteins.

### Example 6

### Separation and Generation/Identification Strategy for Double Stranded DNA Barcodes

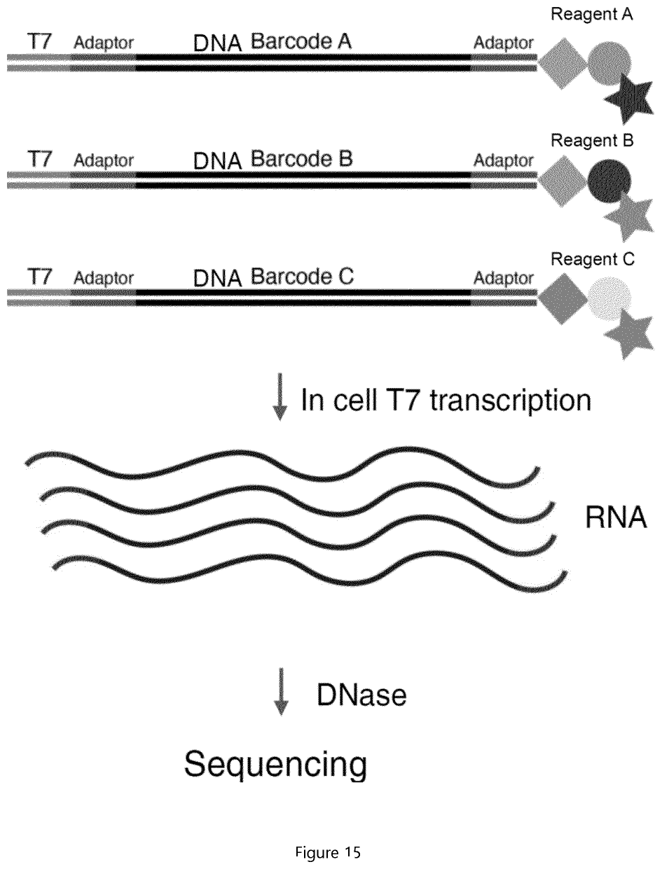

While in-cell amplification is possible using various nucleic acid barcode strategies according to the present invention, the separation and generation/identification strategy might differ depending on the barcode design. The present example depicted in FIG. 15 shows one specific embodiment of a candidate library according to the present invention, wherein the barcode is a dsDNA barcode attached to a reagent (for example a small molecule) of a DNA-encoded library (DEL). Said barcode comprises a T7 promoter/identification sequence, a sequencing adapter for high throughput sequencing, the identifying, unique barcode sequence, and is covalently attached to a reagent identifiable by said barcode sequence. Additionally, said barcode can further comprise attached molecules useful for pulldown of the barcode such as biotin.

The candidate library is first contacted with target cells or tissues to allow cellular uptake and localization of members to various subcellular compartments. Said target cells express a polymerase able to recognize said barcodes. Expression might be limited to the desired subcellular compartment or compartments to ensure amplification of barcodes able to penetrate said desired compartment or compartments, while barcodes not locally overlapping with said polymerase are not amplified.

Following in-cell amplification by T7 polymerase of the barcode of a member of the DEL in desired subcellular compartments according to the invention, the generated T7 transcripts comprise RNA molecules, therefore carrying the information of the barcode sequence, which allows the identification of the chemical reagent attached to the original DNA barcode. However, said sequence is identical to the coding strand of the template dsDNA sequence of the barcode. Reverse transcription of said transcript would result in indistinguishing DNA molecules. Therefore, distinguishing between the transcript molecules and the original dsDNA barcode by sequencing may be challenging.

According to one embodiment of the invention, the original DNA barcode templates can be digested using Dnases before reverse transcription of the in-cell amplification products. The in-cell amplification products are RNA transcripts and therefore not affected by Dnase treatment. Additionally, or alternatively, remaining DNA barcodes can be removed using a purifying molecule attached to said barcode such as biotin by methods known in the art such as affinity chromatography. The affinity chromatography removal step can proceed the Dnase treatment or vice versa. The digestion and pull-down of template barcode DNA molecules allows the removal of both successfully entered barcodes that didn't reach the desired subcellular compartments as well as unspecific binding barcodes attached to the cell. Additionally said strategy ensures that only barcodes are identified that not only internalized into the target cells but also co-localize with the polymerase in the desired subcellular compartment. Barcodes that internalize but fail to reach said compartment are therefore removed from consideration further increasing signal to noise ratio (FIG. 15).

The barcode and separation strategy depicted in FIG. 15 was tested and evaluated in order to estimate the ability to identify T7 transcripts of in-cell amplified barcodes.

A double stranded DNA barcode containing a biotinylated T7 promoter was co-transfected into cells together with a T7 RNA polymerase (T7 RNAP) expressing vector, or into control cells, together with irrelevant DNA plasmid that do not express a T7 RNA polymerase (empty vector). Following in-cell barcode amplification by T7 RNAP, RNA was extracted and non-amplified DNA barcodes were depleted using streptavidin beads. Non-amplified barcodes were further depleted by Dnase digestion. The resulting T7 products were reverse-transcribed into cDNA, and were subjected to qPCR quantitation.

In more detail, all barcode oligos and T7 promoter were synthesized by Eurogentec and IDT. 293T cells (target cells) were co-transfected either with barcodes and T7 RNA polymerase (T7 RNAP+) or as a negative control (T7 RNAP−) with barcodes and irrelevant DNA plasmid, using Lipofectamine 3000 (ThermoFisher) according to the manufacturer recommendations. (3 biological replicates each condition). All barcodes were double stranded. Cells were incubated with the barcodes for 36 hours, after which, cells were thoroughly washed with fresh media and with PBS to remove non-internalized barcodes. Total RNA was isolated using Zymo Quick RNA Miniprep kit (manufacturer recommendations).

For measuring in-cell barcode amplification: the original double-stranded library was depleted using Dynabeads™ MyOne™ Streptavidin C1 magnetic beads (Invitrogen). RNA was further treated with Turbo Dnase I (Thermofisher), and re-purified using Zymo RNA Clean & Concentrator 5 kit. RNA was then used for a strand-specific reverse transcription using Superscript III (Invitrogen, manufacturer standard protocol). cDNA was used for qPCR quantification with custom TaqMan primers that target the cDNA product of in-cell T7-amplified barcodes.

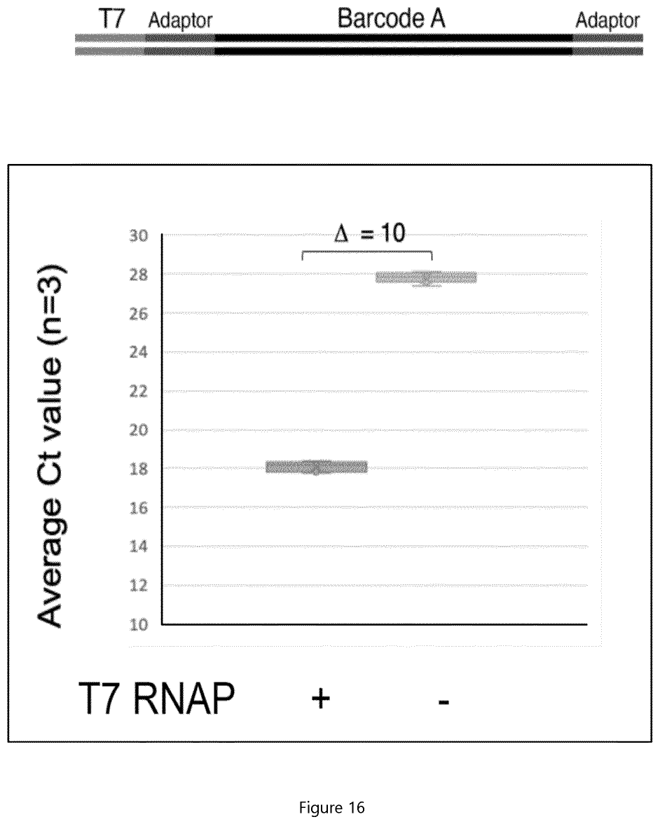

The results are quantified in FIG. 16. The data shows 10 PCR cycles difference between cDNA originating from T7 RNAP expressing cells and control cells not expressing T7. Standard deviations of 3 biological replicates are shown. Therefore, in-cell amplification of barcodes by T7 polymerase increased detection by over 1000-fold in comparison to barcodes that did not amplify due to lack of polymerase. These data clearly demonstrate the ability of separating and identifying dsDNA barcode-derived transcripts as a product of in-cell amplification according to the invention.

### Example 7

### Separation and Generation/Identification Strategy for Single-Stranded DNA Barcodes Comprising a Double Stranded T7 Promoter

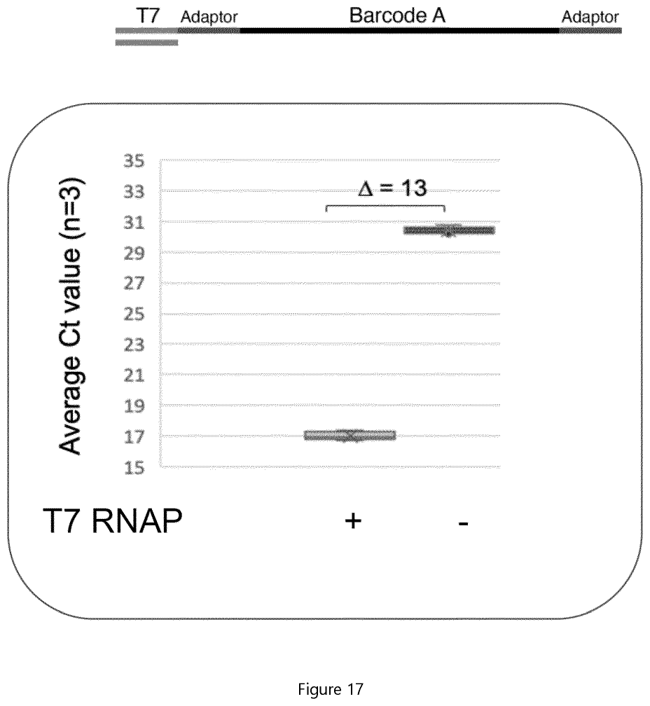

In some embodiments the barcode comprises a hybrid nucleic acid with single and double stranded stretches. The following example depicts a particular embodiment of the present invention, wherein the nucleic acid barcode is a single-stranded DNA barcode further comprising a double stranded T7 promoter (FIG. 17 top).

A single stranded DNA barcode containing a double stranded T7 promoter was transfected into cells that were pre-transfected with a T7 RNA polymerase (T7 RNAP) or into control cells that were pre-transfected with an empty vector. Following in-cell barcode amplification by T7 RNAP, RNA was extracted and subjected to Dnase digestion. A strand-specific reverse transcription primer targeting the T7 product was used to create cDNA. The resulting cDNA was subjected to qPCR quantitation.

In more detail, all barcode oligos and T7 promoter were synthesized by Eurogentec and IDT. 293T cells (target cells) were transfected either with T7 RNA polymerase (T7 RNAP+) or a negative control empty vector (T7 RNAP−) using Lipofectamine 3000 (ThermoFisher) according to the manufacturer recommendations. Following 6 hours incubation, cells were thoroughly washed with fresh media to remove the transfection reagent. Barcode were then added to cells for cellular uptake (3 biological replicates each condition). All barcodes were single stranded, contained cholesterol-TEG for cellular delivery, and pre-annealed to an 18 nt long primer to create a double-strand T7 promoter before addition to cells. Cells were incubated with the barcodes for 36 hours, after which, cells were thoroughly washed with fresh media and with PBS to remove non-internalized barcodes. Total RNA was isolated using Zymo Quick RNA Miniprep kit (manufacturer recommendations).

For measuring in-cell barcode amplification: RNA was treated with Turbo Dnase I (Thermofisher), and re-purified using Zymo RNA Clean & Concentrator 5 kit. RNA was then used for a strand-specific reverse transcription using Superscript IV (Invitrogen, manufacturer standard protocol). cDNA was purified using Dynabeads™ MyOne™ Streptavidin C1 magnetic beads (Invitrogen) and used for qPCR quantification with custom TaqMan primers that target the cDNA product of in-cell T7-amplified barcodes.

The data depicted in FIG. 17 bottom shows over 13 PCR cycles difference between cDNA originating from T7 RNAP expressing cells and control cells not expressing T7. Standard deviations of 3 biological replicates are shown.

As shown in this example, the method according to the present invention is able to distinguish between T7 transcripts and original barcode molecules and shows a high signal to noise ratio necessary for the identification of desired members of the library localizing to the desired subcellular compartment. In fact, in-cell amplification products showed over 10,000-fold increased detection levels compared to the original barcode molecules.

### Example 8

### Barcode Design Comprising Partially Single-Stranded DNA Barcode

In a particular embodiment, the barcode according to the invention can comprise hybrid molecules of single and double stranded nucleic acids.

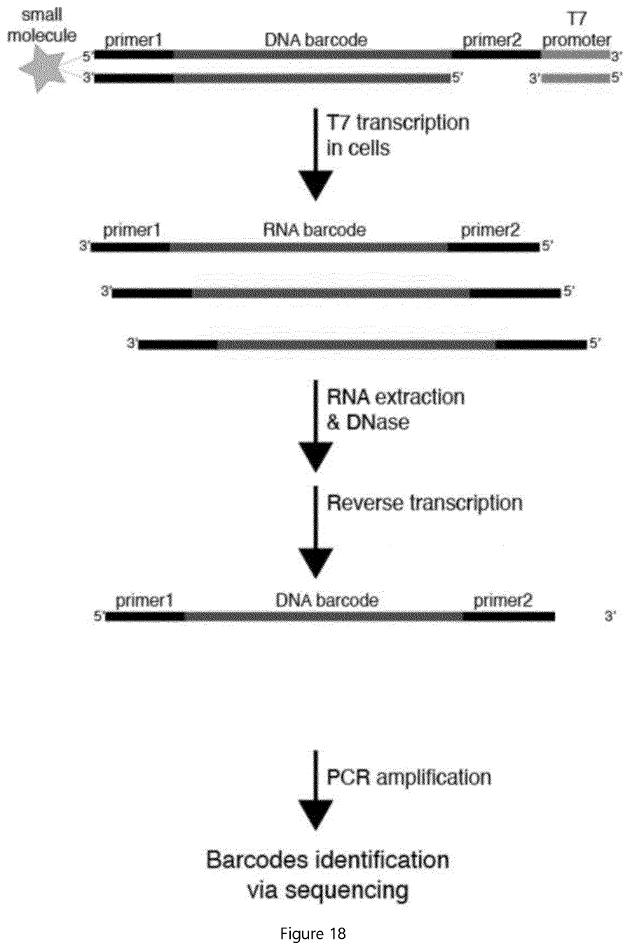

As shown in FIG. 18 a barcode may comprise an RNA polymerase promoter (e.g., T7 promoter) attached to the unique barcode sequence of a DNA to enable signal amplification inside cells that express the cognate polymerase (e.g., cells expressing T7 polymerase). The barcode is partially single-stranded and partially double-stranded. The use of hybrid barcodes with partial single and double stranded stretches allows distinguishing between in-cell amplification products and original barcode molecules. Incorporation of a single stranded primer sequence in the template strand of the original barcode molecule, which is not present in the coding strand enables the identification of in-cell amplification products. Said products comprise the reverse complement sequence of the coding strand, which allows to clearly distinguish between the products and the original molecule by means such as PCR amplification or sequencing.

Following in-cell amplification, the original library is eliminated via enzymatic and/or chemical treatment (e.g., using Dnase digestion) that will degrade the original barcode but not its amplification products (e.g., RNA transcripts). The resulting RNA is reverse transcribed using a strand-specific primer. The resulting in-cell amplification cDNA products are subjected to PCR amplification and identification (e.g., via sequencing).

### Example 9

### Identification of Cell-Internalizing Barcodes

The method of the present invention allows the identification of library members able to internalize into target cells. The following example illustrates the ability of the method to distinguish between library members able to internalize and the ones without these internalizing abilities.

In order to test the ability to distinguish between internalizing and non-internalizing library members, single-stranded or double-stranded barcodes were optimized for cell entry by coupling a cholesterol tag or were not optimized for cell entry (no coupled reagent attached) and incubated with cells that were pre-transfected with T7 RNA polymerase. Following extraction and elimination of non T7-replication products (as described in previous examples 7-9 for single and double stranded barcodes), T7 replication products were quantified via RT-qPCR.

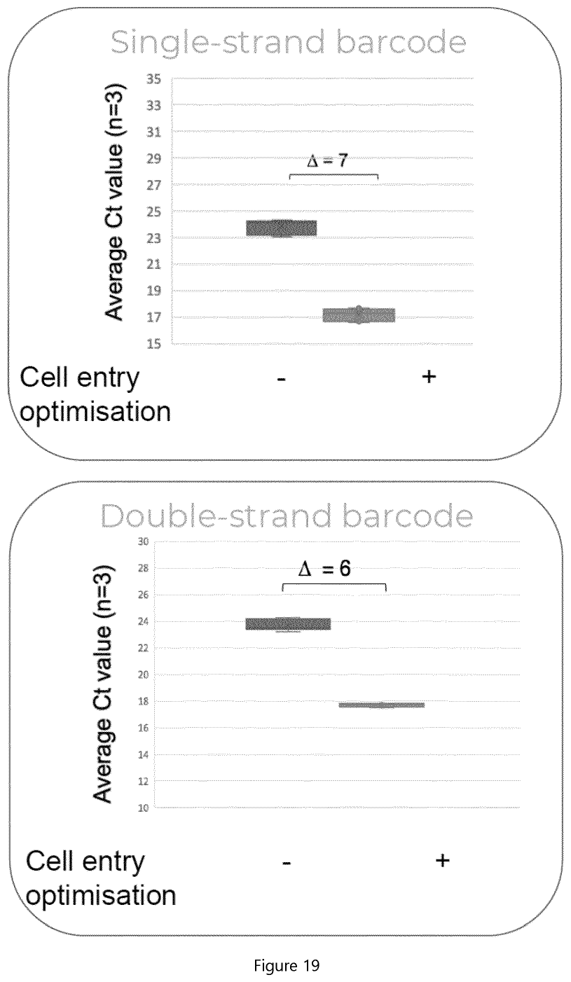

In more detail, all barcode oligos and T7 promoter were synthesized by Eurogentec and IDT. 293T cells (target cells) were transfected either with T7 RNA using Lipofectamine 3000 (Thermofisher) according to the manufacturer recommendations. Following 6 hours incubation, cells were thoroughly washed with fresh media to remove the transfection reagent. Either unconjugated barcode (Cell entry optimization-) or Cholesterol-TEG conjugated barcode (Cell entry optimization+) were then added to cells for cellular uptake (3 biological replicates each condition). Barcodes were either single stranded, and pre-annealed to an 18 nt long primer to create a double-strand T7 promoter before addition to cells (FIG. 19 top) or double stranded (FIG. 19 bottom). Cells were incubated with the barcodes for 36 hours, after which, cells were thoroughly washed with fresh media and with PBS to remove non-internalized barcodes. Total RNA was isolated using Zymo Quick RNA Miniprep kit (manufacturer recommendations.

For measuring in-cell barcode amplification: In the case of single-stranded barcodes, RNA was treated with Turbo Dnase I (Thermofisher), and re-purified using Zymo RNA Clean & Concentrator 5 kit. RNA was then used for a strand-specific reverse transcription using Superscript IV (Invitrogen, manufacturer standard protocol). cDNA was used for qPCR quantification with custom TaqMan primers that target the cDNA product of in-cell T7-amplified barcodes. In the case of double-stranded barcodes, the original double-stranded library was depleted using Dynabeads™ MyOne™ Streptavidin C1 magnetic beads (Invitrogen). RNA was further treated with Turbo Dnase I (Thermofisher), and re-purified using Zymo RNA Clean & Concentrator 5 kit. RNA was then used for a strand-specific reverse transcription using Superscript IV (Invitrogen, manufacturer standard protocol). cDNA was used for qPCR quantification with custom TaqMan primers that target the cDNA product of in-cell T7-amplified barcodes.

The results shown in FIG. 19 illustrate the difference of signal between cell-internalizing barcodes and non-internalizing barcodes. Barcodes able to enter the cell were readily detected by qPCR, while non-internalizing barcodes showed lower signal. Single stranded barcodes that were optimized for cell entry showed about 128 times higher signal compared to barcodes without any optimization for cell entry. Similarly, double stranded barcodes optimized for cell-entry showed about 64 times more signal compared to non-optimized barcodes. These data illustrate the high signal to noise ratio between cell-internalizing barcodes compared to non-internalizing barcodes of different barcode design according to the invention and further support the applicability of the method to identify reagents coupled to barcodes that enter cells with high throughput.

### Example 10

### Identification of Bio-Available Barcodes

A major advantage of the present invention over the state-of-the-art is the ability to identify library members that not only internalize into target cells but also localize to desired subcellular compartments such as the cytoplasm, mitochondria, nucleus, or other organelles.

In order to test the ability of the method of the present invention to distinguish and identify barcodes that internalize into target cells but fail to reach a desired subcellular compartment and barcodes able to internalize and localize to said desired compartment, cells were incubated with barcodes fused to tags known to facilitate transport of cargo barcodes to predetermined subcellular compartments. Barcodes were either coupled to reagents known to internalize into target cells but localize to the mitochondria (non-bioavailable) or to reagents known to internalize and localize to the cytoplasm (bioavailable). The desired subcellular compartment according to the invention is the compartment expressing the polymerase able to recognize the barcodes (here cytoplasm).

Cells, expressing T7 polymerase in the cytoplasm, were incubated with either barcodes not optimized for cell entry (nucleic acid barcode only), barcodes optimized for cell entry and cytoplasmic bioavailability (barcode coupled to cholesterol; see also example 10), or barcodes capable of cell entry but not cytoplasmic bioavailable (barcode coupled to a tetramethylindo (di)-carbocyanine, Cy3). Cytoplasmic bioavailable barcodes were coupled to cholesterol, known to enter the cytoplasm, while non-bioavailable barcodes were coupled to Cy3 known to localize to the mitochondria. Following incubation, the cells were extensively washed to remove non-internalizing barcodes, and their nucleic acid content was extracted. In-cell amplification products were reverse transcribed and quantified using qPCR.

In more detail, all barcode oligos and T7 promoter were synthesized by Eurogentec and IDT. 293T cells (target cells) were transfected with T7 RNA polymerase using Lipofectamine 3000 (Thermofisher) according to the manufacturer recommendations. Following 6 hours incubation to allow expression of the T7 RNAP, cells were thoroughly washed with new media to remove the transfection reagent. Either unconjugated barcode, Cholesterol-TEG conjugated barcode or Cy3 conjugated barcode were then added to cells for cellular uptake (3 biological replicates each condition). All barcodes were single stranded, and pre-annealed to an 18 nt long primer to create a double-strand T7 promoter before addition to cells. Cells were incubated with the barcodes for 36 hours, after which, cells were thoroughly washed with new media and with PBS to remove non-internalized barcodes. Total RNA was isolated using Zymo Quick RNA Miniprep kit (manufacturer recommendations, but the optional Dnase step was omitted).

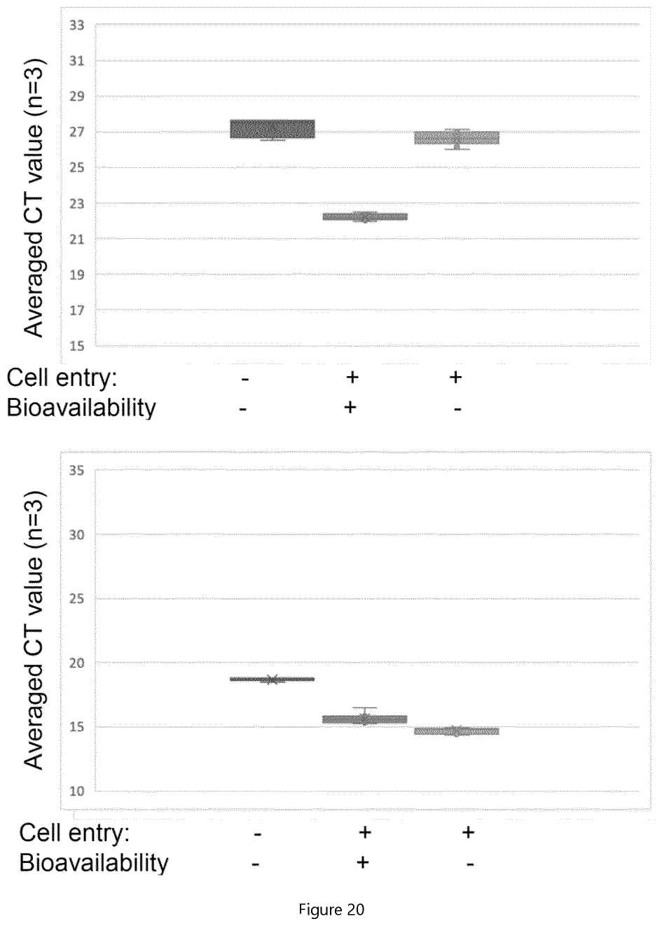

For measuring in-cell barcode amplification (FIG. 20 top): RNA was treated with Turbo Dnase I (Thermofisher), and re-purified using Zymo RNA Clean & Concentrator 5 kit. RNA was then used for a strand-specific reverse transcription using Superscript IV (Invitrogen, manufacturer standard protocol) and a primer that targets the T7 product (which is reverse-complement to the original DNA barcode. cDNA was used for qPCR quantification with custom TaqMan primers that target the cDNA product of in-cell T7-amplified barcodes.

For measuring the cellular uptake of the original DNA barcodes without in-cell amplification-which represents the state of the art (FIG. 20 bottom): Following RNA extraction from cells, but prior to the Turbo Dnase I treatment, the RNA/DNA was taken directly to qPCR quantification using primers that target the original DNA barcodes. (Note: Zymo Quick Rna Miniprep kit used here results in purifying both RNA and small size DNA when the optional Dnase step is omitted).

The data presented in FIG. 20 illustrate signal intensity of barcodes optimized for cell entry (cell entry+) and able to localize to the subcellular compartment expressing T7 polymerase (bioavailability+). Averaged Ct value (n=3). qPCR quantitation of the T7 amplification products demonstrated stronger signal originating from barcodes optimized for cell entry and cytoplasmic bioavailability, while barcodes that entered the cell but did not localize to the desired compartment showed similarly low levels of signal compared to barcodes unable to enter the cell (FIG. 20 top).

Additionally, data is provided showing the ability of the method of the present invention to distinguish between the original barcode molecules and in-cell amplification products during the identification step. FIG. 20 bottom shows quantification of nucleic acids content for which reverse transcription and depletion of the original library were omitted. Therefore, nucleic acids quantified correspond to the original DNA barcode rather than the in-cell amplification products, and representing the state of the art in aptamer and DEL screens. These data show that, while there is a higher detection rate of barcodes able to enter the cell (cell entry+) compared to barcodes not able to enter and washed off during the procedure, simply probing the original barcode molecules, as for instance common practice in Cell-SELEX approaches, does not allow the distinguishing between localization to desired subcellular compartments. Also, the data provided further shows the lower signal to noise ratio of barcodes able to enter the cells (signal) and non-entering barcodes (noise) for probing the original barcode molecules rather than the in-cell amplification products.

These data illustrate the advantages of the present invention. Not only allows the method to distinguish between barcodes or barcodes coupled to reagents able to enter a cell or failing to do so, but also between barcodes localizing to a desired subcellular compartment. The increase of the signal to noise ratio compared to state-of-the-art methods further allows more confidence in lead identification as well as a fast, more efficient high throughput screening procedure.

### Example 11

### Identification of Library Members Able to Localize to Desired Compartment

The present method of the invention is able to identify/generate library members, such as DEL members able to localize to desired subcellular compartments, while allowing to exclude members not able to localize to said compartment.

In order to demonstrate these abilities, previous experiments demonstrated the ability to distinguish between barcodes able to enter a cell, barcodes that do not enter and barcodes that localize to a desired subcellular compartment.

The present example further demonstrates that said method according to the invention is scalable and allows the screening of large numbers of members of a library according to the invention thereby increasing throughput testing and decreasing time commitment.

In order to demonstrate the high throughput abilities of the method according to the invention, a highly complex library according to the invention was assembled and contacted with target cells expressing a polymerase able to identify barcodes comprising the library members. Following incubation and allowing cell penetration and in-cell amplification of barcodes localizing to the subcellular compartment expressing said polymerase, in-cell amplification products were purified and sequenced to identify barcodes able to reach the desired compartment. As a positive control, barcodes with a known unique sequence were coupled to reagents able to localize to the desired compartment. Said positive controls were spiked in along with the other members of the library.

A library of 20-nt long barcodes of random sequences, comprising a biotinylated double-stranded T7 promoter, was spiked in with 5 positive control barcodes of known sequences that were conjugated each to cholesterol. The library was introduced to cells that were pre-transfected with a T7 RNAP. Following a 36-hour incubation, cells were extensively washed to remove non-internalizing barcodes, and nucleic acid content was extracted. For representing a T7 system off state (probing the original barcode molecules): the recovered DNA barcodes were PCR amplified and sequenced without further purification procedures and without reverse transcription of in-cell amplified RNA products, therefore primarily comprising the original barcode molecules, and representing the state of the art in DEL and SELEX screens. For representing a T7 system on state (e.g., probing primarily the in-cell amplification products): the original barcode molecules were depleted using streptavidin beads (utilizing the biotin tag attached to barcodes) and Dnase digestion thereby removing the original barcode molecules, after which the T7 amplification progenies of internalized barcodes were reverse transcribed, PCR amplified and sequenced.

In more detail, all barcode oligos and T7 promoter were synthesized by Eurogentec and IDT. A barcode library that contains a random 20N sequence was spiked-in with 5 barcodes of a known sequence that were conjugated each with Cholesterol-TEG for cellular delivery. The cholesterol conjugated barcodes were spiked-in at 1:10,000 dilution (1 nmol barcode library was spiked in with 100 fmol of each cholesterol-TEG barcode). All barcodes contained a T7 double-stranded DNA promoter, a Biotin-TEG, and binding sites for Illumina sequencing primers (TruSeq read 1 and Truseq read2, partial sequences). 293T cells were first transfected with T7 RNA polymerase using Lipofectamine 3000 according to the manufacturer recommendations. Following 6 hours incubation to allow T7 RNAP expression, cells were thoroughly washed with new media to remove the transfection reagent.
of the spiked-in barcode library was then added for cellular uptake (3 biological replicates). Cells were incubated with the barcodes for 36 hours, after which, cells were thoroughly washed with new media and with PBS to remove non-internalized barcodes, and total RNA was isolated using Zymo Quick RNA Miniprep kit (manufacturer recommendations, but the optional Dnase step was omitted).

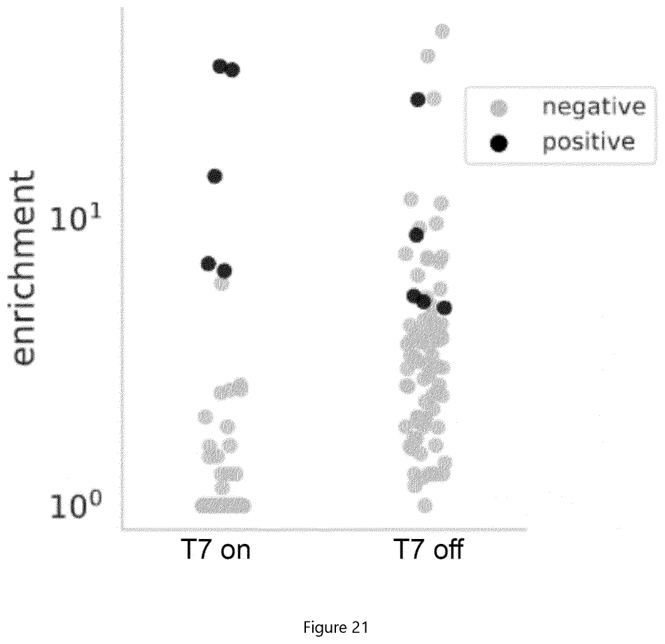

For measuring in-cell barcode amplification (T7 on, FIG. 21 left):

The original DNA library was depleted from the isolated RNA using Invitrogen Dynabeads C1 MyOne Streptavidin magnetic beads. The resulting RNA was further treated with Turbo Dnase I, and re-purified using Zymo RNA Clean & Concentrator 5 kit.

RNA was then used for reverse transcription with Superscript IV (Invitrogen, manufacturer standard protocol) and a primer that targets the expected T7 products.

cDNA was amplified with Phusion High-Fidelity PCR Master Mix (Thermofisher) using Illumina Truseq primers, Size-selected on agarose gel and sequenced on Illumina NovaSeq.

For measuring the cellular uptake of the original DNA barcodes (T7 on, FIG. 21 right):

Following RNA extraction from cells, but prior to the Turbo Dnase I treatment, the RNA/DNA was taken directly to PCR amplification with Phusion High-Fidelity PCR Master Mix (Thermofisher) and Illumina Truseq primers. (Note: Zymo Quick Rna Miniprep kit results in purifying both RNA and small size DNA when the optional Dnase step is omitted). The resulting DNA was size-selected on agarose gel and sequenced on Illumina NovaSeq.

The results were normalized to the sequencing results of the original input library containing the spiked-in barcodes before introducing to cells. The experiment was performed in 3 biological replicates, and the data was merged post-sequencing. The enrichment fold change of each barcode relative to its abundance in the input library is presented in FIG. 21. Shown in the T7 system off state (FIG. 21 right), the 5 most enriched barcodes identified 1 positive control (spiked-in barcode). In the T7 system on state (FIG. 21 left), the 5 most enriched barcodes result in identifying all 5 positive controls.

The data presented in FIG. 21 illustrate the ability of the method according to the invention to identify members of a library able to localize to a desired subcellular compartment. In particular, these data illustrate the scalability of the method allowing to screen a plurality of compounds attached to a barcode for cell entering and localizing properties.

### Example 12

### Screening a Plurality of Compounds for Cell Entering and Localizing Properties

In a second example, a library of 20-nt long barcodes of random sequences, comprising a double-stranded T7 promoter, was spiked in with (i) 5 positive control barcodes that were conjugated each to cholesterol; (ii) 5 negative control barcodes with no conjugate; and (iii) 5 negative control barcodes that were conjugated each to Cy3. All spiked in controls comprised each a barcode of known sequences, and double-stranded T7 promoter with the same design as the bulk-library. The positive and negative controls were spiked-in at either 1:1,000 or 1:100,000 dilutions. The spiked-in library was introduced to cells that were pre-transfected with a T7 RNAP. Following a 36-hour incubation, cells were extensively washed to remove non-internalizing barcodes, and nucleic acid content was extracted. For representing a T7 system off state (probing the original barcode molecules): the recovered DNA barcodes were PCR amplified and sequenced without further purification procedures and without reverse transcription of in-cell amplified RNA products, therefore primarily comprising the original barcode molecules and representing the state of the art in DEL and SELEX screens. For representing a T7 system on state (e.g., probing primarily the in-cell amplification products): the original barcode molecules were depleted using Dnase digestion, after which a strand specific reverse transcription designed to amplify only T7 amplification progenies of internalized barcodes was performed.

In more detail, for 1:1,000 spike in: 1 nmol barcode library was spiked in with 1 pmol of each positive and negative controls (FIG. 23 left panel). For 1:100,000 spike in: 1 nmol barcode library was spiked in with 10 fmol of each positive and negative controls (FIG. 23 right panel). All barcodes contained a T7 double-stranded DNA promoter, and binding sites for Illumina sequencing primers. 293T cells were first transfected with T7 RNA polymerase using Lipofectamine 3000 according to the manufacturer recommendations. Following incubation, cells were thoroughly washed with new media to remove the transfection reagent.
of the spiked-in barcode library was then added for cellular uptake (3 biological replicates for each spike-in dilution). Cells were incubated with the barcodes, after which, cells were thoroughly washed with fresh media and with PBS to remove non-internalized barcodes, and total RNA was isolated using Zymo Quick RNA Miniprep kit (manufacturer recommendations, but the optional Dnase step was omitted).

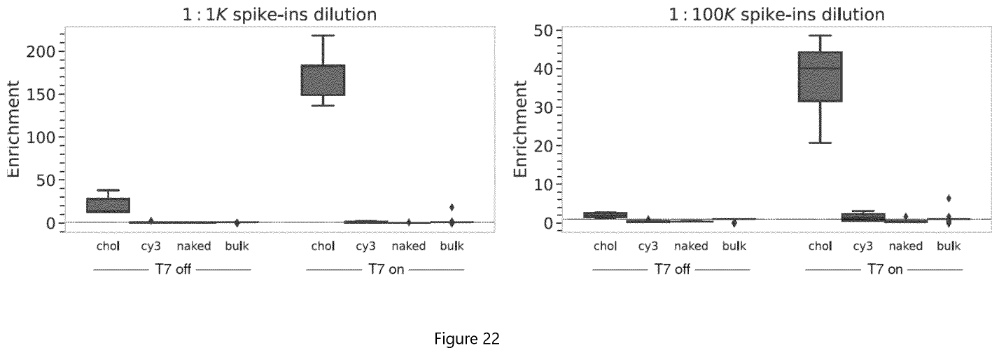

For measuring in-cell barcode amplification (T7 on, FIG. 22):

The original DNA library was depleted from the isolated RNA by treatment with Turbo Dnase I.

RNA was then used for reverse transcription with Superscript IV (Invitrogen, manufacturer standard protocol) and a primer that targets specifically the expected T7 products.

cDNA was amplified with Phusion High-Fidelity PCR Master Mix (Thermofisher) using Illumina Truseq primers, Size-selected on agarose gel and sequenced on Illumina NovaSeq.

For measuring the cellular uptake of the original DNA barcodes (T7 off, FIG. 22): Following RNA extraction from cells, but prior to the Turbo Dnase I treatment, the RNA/DNA was taken directly to PCR amplification with Phusion High-Fidelity PCR Master Mix (Thermofisher) and Illumina Truseq primers. (Note: Zymo Quick Rna Miniprep kit results in purifying both RNA and small size DNA when the optional Dnase step is omitted). The resulting DNA was size-selected on agarose gel and sequenced on Illumina NovaSeq.

The results were normalized to the sequencing results of the original input library containing the spiked-in barcodes before introducing to cells. The experiment was performed in 3 biological replicates, and the data was merged post-sequencing. The enrichment fold change of each barcode relative to its abundance in the input library is presented in FIG. 22.

The data presented in FIG. 22 illustrate the ability of the method according to the invention to identify members of a library able to localize to a desired subcellular compartment. In particular, these data illustrate the scalability of the method allowing to screen a plurality of compounds attached to a barcode for cell entering and localizing properties

What claimed is:

1. A method for generating and/or identifying a reagent, said reagent being able to enter a desired subcellular compartment of a target cell, comprising the following steps:
a. preparing and/or selecting target cells comprising a polymerase localizing to said desired compartment;
b. preparing a candidate library of reagents comprising or consisting of nucleic acid barcodes recognized by said polymerase;
c. contacting said target cells with said library of reagents, wherein at least a subset of reagents interacts with at least a subset of target cells forming cell-reagent complexes;
d. incubating the cell-reagent complexes thus obtained for a period of time at least sufficient to allow at least a subset of said reagents to enter a desired subcellular compartment of a target cell;
e. amplifying the nucleic acid barcode of said subset of reagents of step d) by said polymerase of step a) within the desired subcellular compartment of the target cells and
f. separating the amplified nucleic acid barcodes of step e) to generate and/or identify said reagent.

2. The method according to claim 1, wherein the amplification products of step f) are chemically different from the nucleic acid barcodes of step b), which enables their specific separation.

3. The method of claim 2, wherein the amplification products differ from the nucleic acid barcodes by sequence length, sequence orientation, presence of an affinity tag, and/or nucleic acid class.

4. The method according to any of claims 1 to 3, wherein the target cells are selected from the group of primary cells, cancer cells, immune cells, organs organoids, organ-on-a-chip, or combinations thereof.

5. The method according to any of claims 1 to 4, wherein said polymerase is selected from the group of T3, Sp6, T7 RNA polymerase, Phi29 DNA polymerase, Syn5, or alphavirus replicase.

6. The method according to claim 5, wherein said polymerase is T7 RNA polymerase.

7. The method according to any of claims 1 to 6, wherein said candidate library of reagents is selected from the group of DNA-encoded libraries, aptamer libraries, oligonucleotide libraries, polypeptide libraries, peptide libraries, antibody libraries, nanobody libraries, carbohydrate libraries, lipid libraries or combinations thereof.

8. The method according to any of claims 1 to 7, wherein the desired compartment is selected from the group of cytoplasm, nucleus, Golgi apparatus, endoplasmic reticulum, mitochondria, or combinations thereof.

9. The method according to any of claims 1 to 8, wherein said barcode can identify the chemical identity of the attached reagent.

10. The method according to any of claims 1 to 9, wherein said barcode comprises at least one amplification initialization element.

11. The method according to any of claims 1 to 10, wherein said amplification initialization element is a T7 promoter.

12. The method according to any of claims 1 to 11, wherein said barcode is chemically attached to reagents, form electrostatic interactions with the reagent, and/or is encapsulated by the reagent.

13. The method according to any of claims 1 to 12, wherein said reagent simultaneously is the barcode.

14. The method according to any of claims 1 to 13, wherein the barcode further comprises a reverse transcription primer site.

15. The method according to any one of claims 1 to 14, further comprising step g):
i. preparing a new candidate library of reagents from the identified reagents of step f); and
ii. repeating steps a) and c) to f) using said newly prepared candidate library of reagents,
wherein step g) is repeated at least n times,
wherein n is an integer between 0 and at least 1.

16. The method according to any one of claims 1 to 15 further comprising identifying the reagent of step f) thus obtained by sequencing.

17. The method according to any one of claims 1 to 16, wherein the reagent identified in step f) is further modified or optimized using directed evolution, mutagenesis, or chemical modification.

18. The method according to any of claims 1 to 17, wherein the incubation of the cell-reagent complexes is carried out at a temperature and for a period of time sufficient to allow the reagents to specifically interact with the desired subcellular compartment of the target cell.

19. A delivery reagent comprising a reagent obtainable by the method according to any of claims 1 to 18 and capable of penetrating a desired subcellular compartment of a target cell.

20. A delivery reagent according to claim 19, wherein said delivery reagent is an, oligonucleotide, aptamer, a small molecule, a peptide, a polypeptide a lipid, a Lipid Nano Particle (LNP), a carbohydrate, or a combination thereof.

21. A delivery reagent according to any claims 19 and 20, wherein the reagent is fused to at least one cargo molecule.

22. A delivery reagent according to any one of claims 19 to 21, wherein the cargo molecule is a therapeutic agent, diagnostic agent, imaging agent, or toxin.

23. A pharmaceutical composition comprising a therapeutically effective amount of a delivery reagent according to any of claims 19 to 22.

24. The use of the delivery reagent of any one of claims 19 to 22 as a medicament or for use in therapy of pulmonary disease.
